(19) United States
(12) Patent Application Publication
(10) Pub. No.: US 2026/0090713 A1
(43) Pub. Date: Apr. 2, 2026

(54) METHOD, USE OF ADAPTED OPTOTYPES AND DEVICE FOR DETERMINING VISUAL ACUITY CHARACTERISTICS OF A SUBJECT

(71) Applicant: Rodenstock GmbH, Muenchen (DE)

(72) Inventors: Adam Muschielok, Miesbach (DE); Stephan Trumm, Muenchen (DE)

(21) Appl. No.: 19/110,481

(22) PCT Filed: Sep. 12, 2023
(86) PCT No.: PCT/EP2023/074965

§ 371 Date: Apr. 25, 2025

(30) Foreign Application Priority Data
Sep. 12, 2022 (DE) 10 2022 209 490.5

(51) Int. Cl. A61B 3/036; A61B 3/00; A61B 3/103; A61B 3/113; A61B 3/18

(52) U.S. Cl. CPC A61B 3/036; A61B 3/0025; A61B 3/1035; A61B 3/113; A61B 3/18

(57) ABSTRACT

A method for determining acuity-of-vision characteristics of a test person with a astigmatic visual defect, including: providing visual defect data for the test person, wherein the visual defect data includes a cylinder axis or axial position of a required optical cylinder correction; selecting a preferential direction such that this preferential direction either corresponds to the cylinder axis which is assigned to the optical cylinder correction, is rotated by 90° to this cylinder axis, or is derived from wavefront data using a point spread function; applying an optical power in the selected preferential direction; displaying an adapted visual sign, which has a directional feature, wherein the adapted visual sign is displayed aligned such that its directional feature is parallel to the preferential direction; and determining acuity-of-vision characteristics of the test person for the selected preferential direction, taking into account a dimension of the directional feature and the applied optical power.

### TECHNICAL FIELD

The invention relates to a method, a use of adapted visual signs and an apparatus for determining acuity-of-vision characteristics of a test person.

### BACKGROUND

The determination of visual acuity as acuity-of-vision characteristics of a test person with a visual defect, in particular astigmatic visual defect, is a central task in optometry. An astigmatic visual defect of the test person can be compensated for by holding up and/or applying an optical cylinder correction to the test person in addition to any optical spherical correction required.

Known methods for determining the visual acuity of a test person with an astigmatic visual defect are regularly lengthy, time-consuming and/or error-prone, since they are often based on active feedback from the test person. Furthermore, to determine the visual acuity, it is necessary to compensate for the test person's astigmatic visual defect by holding up an optical cylinder correction and an optical spherical correction, for example by means of a phoropter or trial frame.

Holding up the optical cylinder correction in front of the trial frame or the phoropter is cumbersome and requires space, which is why, in this case, the trial frame or the phoropter can only be combined with other devices such as an eye tracker in a cumbersome manner.

Without holding up the required cylinder correction in the optical unit used to the test person, the astigmatic visual defect cannot be compensated for.

For this reason, it is often not possible to ascertain the visual acuity of the test person in the relevant range, or only a distorted one, in particular if the test person has a significant cylinder defect.

### SUMMARY

The invention is based on the object of simply and reliably determining acuity-of-vision characteristics of test persons with an astigmatic visual defect.

This object is achieved by the subject matters of the independent claims. Preferred embodiments are the subject matters of the dependent claims.

One aspect relates to a method for determining acuity-of-vision characteristics of a test person who has at least one astigmatic visual defect. Here, providing visual defect data of the test person takes place, wherein the visual defect data includes at least one cylinder axis or axial position of a required optical cylinder correction. Selecting a preferential direction such that this preferential direction either corresponds to the cylinder axis which is assigned to the optical cylinder correction or is rotated by 90° to this cylinder axis takes place. As an alternative, the preferential direction may be derived from wavefront data by means of a point spread function. Applying an optical power at least in the selected preferential direction takes place. Displaying at least one adapted visual sign which has a directional feature takes place, wherein the adapted visual sign is displayed aligned such that the directional feature is arranged in parallel to the preferential direction. Finally, the acuity-of-vision characteristics of the test person are determined for the selected preferential direction, taking into account at least one dimension of the directional feature of the adapted visual sign and the applied optical power.

The acuity-of-vision characteristics of the test person may be determined for one eye of the test person, for both eyes individually, i.e. monocularly, or for both eyes together, i.e. binocularly. Preferably, the acuity-of-vision characteristics are determined monocularly for each eye of the test person individually.

The test person has the astigmatic visual defect and therefore requires an optical cylinder correction which corrects and/or reduces his/her visual defect. Quite regularly, persons with an astigmatic visual defect additionally require an optical spherical correction, which is combined with the optical cylinder correction. These optical corrections can, for example, be integrated into a spectacle lens and/or a contact lens and/or an intraocular lens for the test person.

The applied optical power may be achieved by holding up an optical correction (such as a lens) in front of the respective eye of the test person. With the optical correction, light incident to the test person's eye is manipulated. In this way, the optical corrections may correspond to an optical power held up, in particular an optical power with a spherical and/or cylindrical optical power.

In order to select the preferential direction, the visual defect data of the test person is first required. The visual defect data may have been ascertained as part of a subjective and/or objective refraction, for example. The visual defect data may be available as prescription data for the test person. The method for determining the visual acuity may be integrated into an objective and/or subjective refraction determination and/or may be performed subsequently thereto.

The visual defect data comprises at least the cylinder axis of the required optical cylinder correction. In addition, the visual defect data may also comprise the strength of the required optical cylinder correction. The visual defect data may also comprise a required optical spherical correction, the required optical cylinder correction and/or the cylinder axis of the cylinder assigned to the cylinder correction.

Alternatively, the visual defect data may be based on a wavefront analysis and be based on wavefront data ascertained in this way.

On the basis of the visual defect data, in particular on the basis of the assigned cylinder axis, the preferential direction is selected. As the preferential direction, either the cylinder axis may be used directly or a direction rotated by 90° to this cylinder axis.

If the visual defect data is based on wavefront data, the preferential direction may also be derived from this wavefront data by means of a point spread function. Here, the preferential direction may be derived from a point spread function which is calculated on the basis of the wavefront data. Here, the direction and/or axis of the smallest extension of the point spread function, for example, may be selected as the preferential direction. Here, the direction of least confusion may be selected, e.g. as the direction of the smallest standard deviation of the point spread function.

The cylinder axis is usually arranged in a plane which is approximately perpendicular to a selected gaze direction of the test person. As the gaze direction, for example, a gaze direction of the test person in the position of use, which is defined in the relevant standards, may be selected. The cylinder axis is arranged and/or defined in a plane approximately perpendicular to the position of use. Here, in particular, the cylinder axis may be arranged in a plane in which a spectacle lens and/or a contact lens of the test person is to be arranged. Thus, the cylinder axis may coincide in particular with an axis of an optical cylinder correction which is to be integrated into a spectacle lens and/or a contact lens for the test person.

If the cylinder axis is selected directly as the preferential direction, the preferential direction is arranged approximately in the first principal meridian of the required optical cylinder correction and is additionally arranged approximately perpendicular to the gaze direction of the test person.

If a direction rotated by 90° to the cylinder axis is selected as the preferential direction, the rotation by 90° takes place within a plane which is approximately perpendicular to the gaze direction of the test person. Here, the selected preferential direction may be arranged approximately in the second principal meridian of the required optical cylinder correction and may also be approximately perpendicular to the gaze direction of the test person.

After a preferential direction has been selected as described, the optical power is applied at least in the selected preferential direction. Here, e.g., a rotationally symmetrical optical lens may be held up, e.g. an optical spherical correction. In particular, the optical spherical correction which, according to the visual defect data, at least partially corrects the visual defect of the test person in the selected preferential direction may be applied.

If the cylinder axis of the required optical cylinder correction, i.e. the first principal meridian of the cylinder correction, is selected as the preferential direction, the optical power may be, e.g., an optical spherical correction of the magnitude which corresponds exactly to the required spherical correction contained in the visual defect data, without taking into account the cylinder error of the test person.

If the direction rotated by 90° to the cylinder axis, i.e. the second principal meridian through the cylinder correction, is selected as the preferential direction, e.g., an optical spherical correction with a magnitude which results from the sum of the spherical correction stored in the visual defect data plus the cylinder correction stored therein (e.g. both indicated in diopters) may be applied as the optical power. When calculating the sum, the signs of the required spherical correction and the required cylinder correction must be taken into account.

If the visual defect data contains a required spherical correction of s and a required cylinder correction of z (sometimes also abbreviated as c), a correction with the value s for the first principal meridian and one with the value s+z for the second principal meridian may be applied as the optical power.

The application of the optical power may be effected by holding up the optical power to at least one eye of the test person. This may be done, e.g., by physically holding up the respective optical lens, e.g. by means of a trial frame and/or by means of a refraction unit. In an alternative approach, the application of the optical power is not effected physically, but may be simulated as part of a wavefront simulation. The exact type of application may thus depend on the refraction unit used here.

In order to apply the optical power at least in the selected preferential direction, an optical spherical power is preferably used, e.g. a rotationally symmetrical lens. This produces the desired optical power not only in the selected preferential direction, but also in the entire sphere. Here, an optical spherical power may also be simulated virtually.

With the optical power applied in this way, the visual defect of the test person can be at least partially corrected, at least for the selected preferential direction.

Here, it is remarkable that no optical cylinder correction is required to determine the acuity-of-vision characteristics. For determining the acuity-of-vision characteristics according to the methods, it is sufficient that the acuity-of-vision characteristics are ascertained by applying and/or holding up, e.g., a purely optical spherical correction without requiring an optical cylinder correction.

The application of an optical spherical correction as the optical power is usually easier to realize than the application of an optical cylinder correction, e.g. because no cylinder axis needs to be taken into account for the spherical correction. This simplifies the method by dispensing with optical cylinder corrections.

As the acuity-of-vision characteristics, e.g., at least one visual acuity at the applied optical power, and/or a sensitivity of the test person, and/or a visual acuity-correction value pair, and/or at least one refraction value may be determined. A visual acuity-correction value pair contains information about the visual acuity of the test person when the associated correction is applied as an optical power for at least the selected preferential direction. In this respect, the visual acuity-correction value pair may also contain the associated preferential direction.

Visual acuity determination on the basis of visual signs, such as optotypes, is known in principle. According to the invention, however, not (only) normal and unadapted standard visual signs are used but adapted visual signs which are adapted to the selected preferential direction and thus to the visual defect of the test person. Visual signs which have a directional feature are suitable for this purpose.

Here, the adapted visual sign has a feature with an alignment as the directional feature which the test person is to recognize as part of a visual task. Visual signs having directional features are generally known, such as Landolt rings or the Snellen E. However, Landolt rings, e.g., are aligned as standard such that the gap of the respective Landolt ring is arranged either exactly at 0°, at 90°, at 180° etc.

Deviating from this generally customary arrangement of the gap of the Landolt rings as a visual sign, adapted visual signs are now used whose directional feature is arranged exactly and/or as exactly as possible in parallel to the selected preferential direction. If, e.g., the cylinder axis assigned to the required optical cylinder correction is exactly 12°, or generally exactly the angle α, and if this cylinder axis is selected as the preferential direction, the adapted visual sign is arranged so that its directional feature is displayed exactly at the angle of 12°, generally at the angle α. Thus, the visual sign is adapted exactly to the selected preferential direction for which the applied optical spherical correction corrects the visual defect of the test person well.

The arrangement of the directional feature of the adapted visual sign in the preferential direction enables the test person to recognize this feature of the adapted visual sign even if he/she is not fully, i.e. not also cylindrically, corrected. Even if the test person cannot recognize the adapted visual sign completely sharply because his/her astigmatism is not corrected by an optical cylinder correction, he/she can still recognize at least the directional feature with the visual acuity being sufficient accordingly. Thus, the adapted visual sign enables the test person to recognize at least the directional feature of the adapted visual sign when he/she is optimally and/or at least sufficiently corrected in the preferential direction by the applied optical power.

Finally, the visual acuity and/or the acuity-of-vision characteristics of the test person may be determined for the selected preferential direction, taking into account at least one dimension of the directional feature of the adapted visual sign. The determination of the visual acuity may be effected in the usual way, i.e. depending on the dimension of the directional feature which the test person can still just barely recognize and/or identify at the applied optical power.

In order to ascertain the dimension required for the visual acuity calculation, several adapted visual signs, which differ with regard to one dimension of the directional feature, may be shown to the test person one after the other and/or simultaneously. As part of at least one visual task, the test person may be asked to recognize at least one adapted visual sign.

As part of the visual task and/or a sequence of visual tasks, e.g., the at least one adapted visual sign may be displayed smaller and smaller so that the visual tasks gradually become more difficult. Alternatively, the differently dimensioned adapted visual signs may also be displayed simultaneously. Here, it may be ascertained up to which dimension of the directional feature the test person can still recognize the adapted visual sign.

The test person's visual defect data required in the method may, e.g., correspond to the best correction and/or the best refraction required by the test person to correct his/her visual defect. Additionally or alternatively, the visual defect data may also slightly deviate from the best optical corrections required. For example, the visual defect data may correspond to the data which has been ascertained from an objective refraction measurement for the test person. The objectively ascertained refraction data, at least in the cylinder axis ascertained here, usually corresponds very precisely to the cylinder axis of the cylinder correction actually required by the test person.

In addition to the best correction, e.g., an optical correction somewhat “blurred”, i.e. altered, with respect to the best correction may also be applied as the optical power, for example as part of the determination of the test person's sensitivity. Here, intentionally “worsened” visual defect data may also be used.

In one variant, the applied optical power may be completely independent of the visual defect data of the test person. Here, the dimension of the directional feature of the displayed adapted visual sign may be kept constant, and instead the applied optical power may be varied for this constant dimension until the test person can recognize (or can no longer recognize) the directional feature of the adapted visual sign. In this way, a visual acuity-correction value pair can be ascertained, which may be independent of the (subjectively and/or objectively ascertained) optimal correction.

In principle, however, a visual acuity determination as acuity-of-vision characteristics for the selected preferential direction may be determined most accurately precisely when the optimal optical correction required by the test person in the selected preferential direction is used as the optical power.

The method allows to determine acuity-of-vision characteristics of the test person, such as visual acuity, without holding up and/or applying optical cylinder corrections to the test person. Instead, the adapted visual signs are used, which are precisely adapted to the cylinder axis of the required cylinder correction and can thus make application of optical cylinder corrections superfluous. This allows to perform the determination of the acuity-of-vision characteristics with, e.g., a less expensive device which cannot apply any optical cylinder correction itself.

Furthermore, the method allows to combine the refraction unit used with additional devices since, overall, more space is available for additional devices when the possibility of applying the required cylinder correction can be dispensed with. Thus, e.g., a refractometer, in particular an autorefractometer, may be used as a refraction unit for applying the optical power.

During a visual acuity measurement, the visual defect of the test person can only be corrected for the selected preferential direction, but not for the principal meridian, perpendicular thereto, of the required cylinder correction.

As adapted visual signs, optotypes whose lowest spatial frequencies lie in the direction of the most uncorrected principal meridian, i.e. are aligned in parallel thereto, may be used. Alternatively or additionally, the highest spatial frequencies of the optotypes used may lie in the direction of the best corrected principal meridian, i.e. be aligned in parallel thereto.

When using objective measurements and/or combined objective and subjective measurements to provide the visual defect data, the cylinder axis, i.e. the orientation, may be taken directly from the objective data, since the objective measurement of the cylinder axis is usually very reliable. The objectively measured cylinder, i.e. the objectively measured optical cylinder correction required, may be somewhat reduced in comparison to the measured visual defect data, since the objectively measured cylinder is often not accepted at full strength by the test person. If necessary, the spherical equivalent should be calculated here before adapting the cylinder power, and this should be taken instead of the sphere when calculating the principal meridians.

According to one embodiment, the directional feature of the adapted visual sign has a sequence of light and dark areas which follow one another along the preferential direction. The sequence of light and dark areas may, e.g., be aligned perpendicular to the lines of a hatching or perpendicular to the arrangement of the gap of a Landolt ring. Thus, perpendicular to the gap of a Landolt ring, the dark circular edge is first followed by the light gap and then the dark circular edge again. Thus, the directional feature of a Landolt ring is arranged perpendicular to the gap. In the case of a Snellen E, the directional feature is arranged perpendicular to the three parallel E lines. In general, the directional feature may correspond to a sequence of at least one light area followed by a dark area, preferably at least the interruption of a dark area by a light area or, conversely, at least the interruption of a light area by a dark area. Here, the light and/or dark areas may, e.g., be designed as lines and/or have edges aligned perpendicular to the preferential direction.

According to one embodiment, providing an unadapted standard visual sign having a directional feature takes place. The standard visual sign is rotated in a display plane such that its directional feature is arranged in parallel to the preferential direction. Finally, the standard visual sign thus rotated is displayed as the adapted visual sign. The display may be effected in particular on a screen within a display plane. Here, the unadapted standard visual sign may initially be assumed, which is rotated (e.g. purely mathematically, without being displayed) so that it is arranged so as to be correctly aligned with the selected preferential direction. Thereby, the unadapted standard visual sign is turned into the adapted visual sign. Only after this internal calculation is the adapted visual sign displayed. As the display plane, e.g., the one within which the screen can display the visual signs may be used. The display plane is preferably arranged approximately perpendicular to the gaze direction of the test person and/or approximately in parallel to the selected preferential direction.

By using an initially unadapted standard visual sign having a directional feature, the adapted visual sign, which can be adapted to any cylinder axis of the required optical cylinder correction, can be provided from this one unadapted standard visual sign in a simple manner by rotating it, e.g. around the center of the standard visual sign and/or around another point in the display plane. In addition, the adapted visual sign may be scaled as desired on the display, so that the adapted visual sign can be displayed and/or shown larger or smaller, depending on the visual task currently presented to the test person.

According to one embodiment, the cylinder axis which is assigned to the optical cylinder correction and which is arranged in the first principal meridian of the required optical cylinder correction is selected as a first preferential direction, wherein as the optical power, an optical spherical correction is applied which corrects the visual defect of the test person in the first principal meridian according to the visual defect data, and wherein as acuity-of-vision characteristics, the visual acuity of the test person is determined for this first principal meridian. Alternatively or additionally, a direction rotated by 90° to the cylinder axis is selected as a second preferential direction which is arranged in the second principal meridian of the required optical cylinder correction, wherein as the optical power, an optical spherical correction is applied which corrects the visual defect of the test person in the second principal meridian according to the visual defect data, and wherein as acuity-of-vision characteristics, the visual acuity of the test person is determined for this second principal meridian. If the first preferential direction is selected, exactly the optical spherical correction which is stored in the visual defect data as a required optical spherical correction. If the second preferential direction is selected, i.e. the second principal meridian, the sum of the optical spherical correction stored in the visual defect data plus the stored optical cylinder correction may be selected as the optical spherical correction. This sum corresponds to the correction required by the test person in the second principal meridian. Thus, with the optical spherical correction selected in this way, the visual defect of the test person is corrected relatively well and/or in the best possible way at least in the selected preferential direction, i.e. in the selected principal meridian.

In a further development, the visual acuity of the test person is determined for both the first and the second principal meridian of the required optical cylinder correction and a direction-independent visual acuity is derived therefrom. In other words, both the first preferential direction is selected and, in doing so, the visual acuity of the test person is determined for the first principal meridian, and the second preferential direction is selected and the visual acuity of the test person is determined for the second principal meridian. As a result of the visual acuity determination the result for each principal meridian may initially be indicated. In addition, a direction-independent visual acuity may be derived from these two visual acuity values. The direction-independent visual acuity may be indicated, e.g., as the highest value ascertained during the measurements, as the lowest value, as an arithmetic mean, as a geometric mean, as a harmonic mean, as a logarithmic mean, as a quadratic mean, as a cubic mean, or as a combination of selected ones of the aforementioned values.

According to one embodiment, the visual acuity is only determined for one of the two principal meridians. Here, an excellent principal meridian is selected. As the excellent principal meridian, the principal meridian for which the required optical correction is arranged more in the plus direction, or more in the minus direction, or for which a stronger correction is required in terms of magnitude, or for which the weaker correction is required in terms of magnitude. It is also possible to select the principal meridian which is closer to the vertical in terms of its cylinder axis, or the principal meridian whose cylinder axis is closer to the horizontal.

In general, visual acuity can be understood as the recognizability depending on size, but also the recognizability depending on other parameters which influence the representation, such as contrast. As the visual acuity, a combination of the recognizability depending on the size and the recognizability of the contrast and/or other of these parameters may also be used.

According to one embodiment, a Landolt ring whose gap is displayed rotated by 90° to the selected preferential direction is used as an adapted visual sign. The alignment of the gap rotated by 90° to the selected preferential direction causes the dark-light-dark sequence to be arranged exactly in the selected preferential direction across the gap of the Landolt ring. Thus, the directional feature of the Landolt ring is arranged exactly in parallel to the selected preferential direction and the visual acuity for the selected preferential direction can be easily determined.

According to one embodiment, a Snellen E in which the connecting line connecting the three parallel E-lines is arranged in parallel to the selected preferential direction is used as an adapted visual sign. Here, the directional feature of this Snellen E is the sequence of the three parallel E-lines, e.g. the sequence light (background), dark of the upper line, light of the intermediate space, dark of the central line, light of the intermediate space, dark of the lower E-line, and finally light of the background. This also allows for easily adapting the Snellen E as a standard visual sign to the preferential direction and thus using it as an adapted visual sign.

In a further development, the adapted visual sign is displayed at least once rotated clockwise by 90° to the preferential direction and at least once rotated counterclockwise by 90° to the preferential direction. Here, as part of a visual task, the test person is asked to distinguish these two different rotated adapted visual signs from one another. The test person may be asked, e.g., to distinguish whether the gap of the Landolt ring is directed to the left or to the right if the preferential direction is aligned approximately vertically upwards. This applies similarly to the use of the Snellen E as an adapted visual sign.

According to one embodiment, a hatched area in which the hatching lines are arranged perpendicular to the selected preferential direction is used as an adapted visual sign. For example, a figure may be used as the hatched area, e.g. a circle, a rectangle, a symbol, an animal, letters or the like. The figure is designed to be hatch-filled. Here, the figure preferably does not have any border line which could interfere with the hatching, but is simply a hatch-filled, borderless figure. Here, the lines of the hatching are displayed perpendicular to the preferential direction, since the directional feature, as the relevant feature of the visual sign, is the succession of alternating light and dark areas of the hatching. Here, it may be advantageous if the figure itself is formed to be as uniform as possible and has few details, e.g. is designed as a circle or a square. Figures which are designed to be as simple as possible and have as few details as possible are therefore preferred here. For adjusting the alignment of the directional feature, either the entire visual sign including the hatching may be rotated and/or turned, or only the hatching within the area and/or figure which is kept constant. Here, the hatching may be designed to be binary, i.e. have hard black and/or white edges, or may be designed with a continuous course. For example, the hatching may be designed with a continuous course, e.g. with a sinusoidal intensity course or a similar intensity course.

According to one embodiment, in addition to the adapted visual sign, at least one further visual sign is displayed whose gray value corresponds approximately to an averaged gray value of the adapted visual sign, and the test person is asked to distinguish the displayed visual signs from one another as part of a visual task. Instead of a visual sign with an averaged gray value, a visual sign may also be used whose hatchings are not perpendicular to the selected preferential direction like the adapted visual sign, but rather in parallel to the preferential direction. Such a visual sign appears to the test person as a substantially gray visual sign perpendicular to the preferential direction due to the astigmatic visual defect incorrectly corrected for this hatching alignment. For example, on a display plane as part of a visual task, several such gray visual signs and one adapted visual sign may be displayed, or conversely several adapted visual signs and one such gray visual sign. The test person may be asked to identify which of the displayed visual signs is different from the other visual signs.

According to one embodiment, the applied optical power is varied at least up to a limit refraction for the selected preferential direction from which the test person recognizes the directional feature of the adapted visual sign. Here, the dimension of the displayed adapted visual sign may be kept constant. Here, e.g., an extreme value of the refraction unit used, e.g. ±20 diopters, may be used as a starting value of the applied optical power. Alternatively, a diopter value which deviates by a predetermined deviation of e.g. ±5 diopters from the optical spherical correction actually required according to the visual defect data may also be used as a starting value. After applying the starting value, the applied optical power is varied, e.g. continuously or in fixed steps, until the test person can recognize (or can no longer recognize) the directional feature of the adapted visual sign. The optical power currently applied when recognizing the directional feature of the adapted visual sign, as the limit refraction, corresponds to an optical correction in which the test person has a visual acuity dependent on the dimension of the directional feature of the displayed adapted visual sign. Thus, a visual acuity-refraction value pair are determined as acuity-of-vision characteristics for the preferential direction.

If this method is repeated with at least a second adapted visual sign in which the directional feature is dimensioned differently, a second visual acuity-refraction value pair may be determined which differs from the visual acuity-refraction value pair determined first. From these two different visual acuity-refraction value pairs, e.g., the sensitivity of the test person may be ascertained.

In an alternative embodiment, the dimension of the directional feature of the adapted visual sign is varied at least up to a limit dimension up to which the test person recognizes the directional feature of the adapted visual sign. Here, the applied optical power may be kept constant. As the optical power, e.g., an optical correction may be applied which corrects the visual defect of the test person in the selected preferential direction according to the visual defect data. Here, e.g., an optimal optical correction may be used which has been determined as part of an objective and/or subjective refraction. With this alternative, the dimension of the directional feature of the adapted visual sign can be varied and it can be checked up to which limit dimension the test person can still recognize the directional feature. From this limit dimension, the visual acuity can be determined in a conventional manner. The variation of the dimension of the directional feature of the adapted visual sign can be achieved by displaying differently dimensioned adapted visual signs and/or by varying the size of the displayed adapted visual sign(s). This may be done as part of at least one visual task and/or a sequence of different visual tasks, wherein at least one adapted visual sign is displayed as part of each visual task. In this way, the visual acuity can be determined as acuity-of-vision characteristics for the selected preferential direction for this applied optical power. Here as well, a visual acuity-refraction value pair with the associated preferential direction may be ascertained.

If this method is repeated with at least a second optical power applied in the selected preferential direction, a second visual acuity-refraction value pair may be determined which differs from the visual acuity-refraction value pair determined first. From these two different visual acuity-refraction value pairs, e.g., the sensitivity of the test person may be ascertained.

In general, according to one embodiment, at least one visual acuity and/or at least one sensitivity and/or at least one visual acuity-refraction value pair and/or at least one refraction value may be ascertained as acuity-of-vision characteristics. Here, in particular, a value related to the visual acuity may be ascertained, i.e. at least one visual acuity and/or at least one sensitivity and/or at least one visual acuity-refraction value pair.

Here, the sensitivity may be ascertained depending on a sensitivity metric, e.g. specifically for at least the selected preferential direction. In addition, a sensitivity may also be ascertained for the second preferential direction, i.e. the direction rotated by 90° to the first selected preferential direction. However, a direction-independent sensitivity may also be ascertained (alternatively or additionally). The direction-independent sensitivity may, e.g., be ascertained from the two sensitivities for the first and second preferential directions, or on the basis of two direction-independent visual acuity-refraction value pairs (in which, e.g., the associated direction-independent visual acuity has been determined as an average value of the visual acuity values for the two preferential directions), or on the basis of a sensitivity metric capable of doing so, which may ascertain a direction-independent sensitivity from at least two direction-dependent visual acuity-refraction value pairs.

According to one embodiment, the test person is presented with at least one visual task dependent on the displayed adapted visual sign, which the test person answers by giving active and/or passive feedback. One embodiment of active feedback may be, e.g., that the test person verbally answers a question from an optician and/or another examiner about a visual task. In the same way, active feedback may be given, e.g., by pressing a button and/or a mouse, with a gesture and/or with gaze. The gaze of the test person may be acquired, e.g., by means of an eye tracking unit.

By means of such an eye tracking unit, a passive response, i.e. passive feedback, may also be given. In this way, it is possible to recognize which visual sign the test person is currently fixating by means of the eye tracking unit. Here, it is possible to recognize whether the test person is fixating a visual sign different from the other visual signs, e.g. subconsciously, because he/she has recognized it, or whether the test person is not able to recognize the different visual sign. In principle, visual tasks with passive and active responses may be combined with one another. Preferably, the test person's response, i.e. the feedback, is acquired without intervention of an examiner. Thus, the test person may either actively enter the feedback themselves, for example using a button and/or a mouse-like controller, or it is acquired passively. Dispensing with an examiner as a necessary recipient of the visual task eliminates a possible source of error in the visual acuity determination, namely the human examiner. In addition, costs and/or time can be saved by dispensing with the human examiner.

According to one embodiment, the visual acuity of the test person in the selected preferential direction is determined at two different applied optical powers and a sensitivity of the test person is ascertained therefrom. For example, the visual acuity in the selected preferential direction may be ascertained once with the optimal and/or best optical power for this selected preferential direction, and a second time with an applied optical power different therefrom. This additional optical power may be shifted, e.g., by ±0.5 dpt in comparison to the best power. The sensitivity of the test person may be ascertained from the two visual acuity values which result for the test person at the two different optical powers (i.e. corrections).

In principle, the sensitivity may also be ascertained on the basis of two visual acuity values, neither of which is determined in optimal correction. By means of a mathematical model, the sensitivity of the eye and/or the test person can be calculated from the two visual acuity values ascertained. Thus, it is not absolutely necessary here that the best correction is already known at the time of the visual acuity determination.

Not all of the correction values and/or visual acuity values used to measure sensitivity need to be acquired using the method according to the invention. As part of an autorefractometric and/or aberrometric measurement, e.g., a first visual acuity may be ascertained, e.g. at a given distance from the refraction objectively ascertained therein, and a second visual acuity may be ascertained, e.g. as part of a subjective refraction subsequent thereto, at the best optical correction resulting from the subjective refraction.

However, two or more visual acuity values may also be acquired using the method according to the invention. As part of an autorefractometric and/or aberrometric measurement, e.g., a first visual acuity value may be ascertained, e.g., at the best correction objectively ascertained therein, and a second visual acuity value at a given distance therefrom. In order to calculate the actual visual acuity of the test person, the objective refraction ascertained therein may be used as the best optical correction.

According to one embodiment, a subjective and/or objective refraction is performed and the visual defect data of the test person is derived from the visual defect of the test person ascertained therein. In this way, the visual defect data can be ascertained from an objective refraction, wherein the ascertained optimal optical corrections and the ascertained optimal cylinder axis are used as visual defect data. Alternatively or additionally, a subjective refraction may be performed. Here, the visual defect data may be based on the result of the subjective refraction. Finally, the two results may also be combined and an average value of the objectively ascertained best correction and the subjectively ascertained best correction may be used as visual defect data. In particular as part of a sensitivity determination, optical corrections which deviate from the ascertained best correction may also be used as visual defect data.

In one embodiment, for example, an objective refraction measurement is first performed on the test person and the best optical correction ascertained therein is used as visual defect data. Then, a subjective refraction is performed, wherein while performing the subjective refraction, two visual acuity values are determined using the method according to the invention. The sensitivity is ascertained from these two visual acuity values. Here, in particular, a visual acuity value is determined after completion of the subjective refraction on the basis of visual defect data resulting from the subjectively ascertained best optical correction. In this embodiment, both an objective refraction and a subjective refraction are performed, the visual acuity of the test person is determined and his/her sensitivity is determined. Only a spherical optical correction is applied here for visual acuity determination. An optical cylinder correction is not required.

According to one embodiment, the visual acuity of the test person is determined as the acuity-of-vision characteristics by means of the method and converted into a visual acuity type different therefrom. This conversion may be performed subsequently. The visual acuity is usually dependent on the visual sign used. Since there are different methods for determining the visual acuity, e.g. on the basis of numbers or by means of a grating, e.g. FrACT, the visual acuity values may depend on the determination method used. The visual acuity values dependent on the measurement method may be converted into each other. The conversion may be effected by means of a calibration function which performs the desired conversion.

The calibration function may be determined by means of regression from a data set which contains a plurality of visual acuity values and thus visual acuity types of the same person which were ascertained by different measurement methods (e.g. on the basis of numbers and FrACT). Thus, by means of the data set, a correlation between the two different visual acuity types and/or visual acuity values may be established. In the simplest case, the calibration function may be a function of the visual acuity determined using the method and calculate the visual acuity value which would have resulted on the basis of the other desired determination method as the functional value.

In order to improve the conversion accuracy, the calibration function may depend on further parameters, e.g. the pupil diameter of the person prior to the visual acuity measurement, an orientation of the selected preferential direction, the adapted visual signs used, the uncorrected optical power in one or both principal meridians (e.g. in the best corrected or in the most uncorrected principal meridian), further parameters of the adapted visual signs used (e.g. their contrast), or a combination of some or all of these parameters. The use of the calibration function allows to convert the visual acuity value ascertained with the method into a visual acuity value calculated with other methods, e.g., not ascertaining optotypes provided with a directional feature.

According to one embodiment, the at least one adapted visual sign is displayed so as to be presented without correction and/or without complete correction of the optical cylinder correction required by the test person, but nevertheless sharply for the test person. Here, image elements which appear sharp may be displayed to the test person, despite the correction of the astigmatic visual defect being absent or incomplete. For this purpose, the at least one image element is displayed as an adapted visual sign which is aligned with the preferential direction so as to be perceived as sharp by the test person. This is done without and/or without complete correction of the optical cylinder correction required by the test person. By the at least one directional feature of the adapted visual sign being displayed aligned in parallel to the preferential direction, the test person can perceive it as sharp. In this way, the adapted visual sign can still be perceived as sharp despite the lack of (complete) cylinder correction and can be fixated better and/or more easily by the test person than a visual sign displayed as blurred. This may be advantageous, e.g., when measuring the ability to accommodate, since a viewed object becoming blurred is perceived more quickly by test persons with uncorrected astigmatism.

If, e.g., an image of a hot air balloon is used as a target of a visual task, the direction in which stripes run on the hot air balloon (cf. also FIGS. 6 and 7) may be displayed as being arranged in parallel or perpendicular to the selected preferential direction. The test person can perceive the image of the hot air balloon with the stripes aligned in this way as a directional feature as sharp even without cylinder correction and therefore more quickly. Here, the stripe pattern of the hot air balloon may be displayed like a hatching, adapted to the selected preferential direction.

This allows to set a visual task with sufficient accuracy, e.g. when determining a refraction value and/or a visual acuity value without correcting the astigmatism of the test person (or with incompletely corrected astigmatism).

One aspect relates to a use of adapted visual signs, each of which has a directional feature which is arranged in parallel to a preferential direction which either corresponds to a cylinder axis or axial position which is assigned to an optical cylinder correction required by a test person, or which is rotated by 90° to this cylinder axis, or the preferential direction may be derived from wavefront data by means of a point spread function to determine acuity-of-vision characteristics of the test person for the selected preferential direction, taking into account at least one dimension of the directional feature of the adapted visual sign.

The use of the adapted visual sign may in particular be effected as part of the method described above. Therefore, all explanations of the method may also relate to the use and vice versa.

One aspect relates to an apparatus for determining acuity-of-vision characteristics of a test person who has at least one astigmatic visual defect. The apparatus has a selection module which selects a preferential direction, wherein this preferential direction either corresponds to a cylinder axis or axial position which is assigned to an optical cylinder correction required by the test person, or is rotated by 90° to this cylinder axis. Alternatively, the preferential direction may be derived from wavefront data by means of a point spread function. A refraction unit is configured to apply an optical power to the test person at least in the selected preferential direction. The refraction unit may, e.g., be designed as an aberrometer and/or as a refractometer and/or apply a rotationally symmetrical lens as a spherical correction as the optical power. A display module has a display and displays at least one adapted visual sign having a directional feature on the display such that the directional feature of the adapted visual sign is arranged in parallel to the preferential direction. An acuity-of-vision characteristics determination module determines the acuity-of-vision characteristics of the test person for the selected preferential direction, taking into account at least one dimension of the directional feature of the adapted visual sign and the applied optical power.

The apparatus may be used, e.g., to perform the method described above and/or to use the adapted visual signs described above. Therefore, all explanations of the apparatus also relate to the method and the use and vice versa.

As a dimension of the directional feature, for example, a distance between hatching lines, a contrast strength, a thickness of lines and/or a width of gaps may be used.

According to one embodiment, the apparatus has an eye tracking unit which tracks at least one eye of the test person when displaying the at least one adapted visual sign. By means of the eye tracking unit, on the one hand, a gaze direction of the test person may be ascertained and, on the other hand, an active and/or passive feedback of the test person may be registered as a response to a visual task.

Within the scope of the present invention, the terms “substantially” and/or “approximately” may be used to include a deviation of up to 5% from a numerical value following the term, a deviation of up to 5° from a direction following the term and/or from an angle following the term.

Terms such as up, down, above, below, lateral, etc.—unless otherwise stated—refer to the earth's reference system in an operating position of the subject matter of the invention.

### DESCRIPTION OF THE DRAWINGS

The invention is described in more detail below with reference to exemplary embodiments shown in figures. Here, identical or similar reference signs may indicate identical or similar features of the embodiments. Individual features shown in the figures may be implemented in other exemplary embodiments. Shown are:

FIG. 1A exemplary embodiments of displayed visual signs for determining a visual acuity;

FIG. 1B the visual impression, resulting for the visual signs shown in FIG. 1A, of a test person who is corrected for the second principal meridian of his/her cylindrical visual defect with an optical spherical correction;

FIG. 1C the visual impression, resulting for the visual signs shown in FIG. 1A, of a test person who is corrected for the first principal meridian of his/her cylindrical visual defect with an optical spherical correction;

FIG. 2A exemplary embodiments of adapted visual signs for a visual task for a test person who is corrected for the second principal meridian of his/her cylindrical visual defect with an optical spherical correction;

FIG. 2B the visual impression, resulting for the visual signs shown in FIG. 2A, of a test person who is corrected for the second principal meridian of his/her cylindrical visual defect with an optical spherical correction;

FIG. 3A exemplary embodiments of adapted visual signs for a visual task for a test person who is corrected for the second principal meridian of his/her cylindrical visual defect with an optical spherical correction;

FIG. 3B the visual impression, resulting for the visual signs shown in FIG. 3A, of a test person who is corrected for the second principal meridian of his/her cylindrical visual defect with an optical spherical correction;

FIG. 4A the visual impression, resulting for differently dimensioned visual signs, of a test person of adapted visual signs, wherein the test person is corrected for the second principal meridian of his/her cylindrical visual defect with an optical spherical correction;

FIG. 4B the visual impression, resulting for differently dimensioned visual signs, of a test person of adapted visual signs, wherein the test person is corrected for the second principal meridian of his/her cylindrical visual defect with an optical spherical correction;

FIG. 5A exemplary embodiments of adapted visual signs for a visual task for a test person who is corrected for the second principal meridian of his/her cylindrical visual defect with an optical spherical correction;

FIG. 5B the visual impression, resulting for the visual signs shown in FIG. 5A, of a test person who is corrected for the second principal meridian of his/her cylindrical visual defect with an optical spherical correction;

FIG. 6 an exemplary image or photograph which conveys a sense of distance to the viewer;

FIG. 7 the image or photograph of FIG. 6 with exemplary adapted visual signs integrated in the image or superimposed on the image; and

FIG. 8 a graph of the accommodation width depending on age (Duane curve).

### DETAILED DESCRIPTION

The figures show exemplary embodiments of visual signs and the visual impression resulting therefrom in a test person with an astigmatic visual defect. Here, the test person is assumed to have a visual defect in the sphere of +2.75 dpt and, in addition, an astigmatism of −3.0 dpt at a cylinder axis of 12°. This visual defect data is to be understood as an example and the following exemplary embodiments are generally applicable to test persons who have a visual defect in the sphere of s and, in addition, an astigmatism of z at a cylinder axis of α.

The visual defect of the test person may be acquired as part of a subjective and/or objective refraction. This results in visual defect data in which the spherical and astigmatic visual defect including the cylinder axis is contained, e.g. at least the set of values {s; z; α}, in the example the set of values {+2.75 dpt; −3.0 dpt; 12°}.

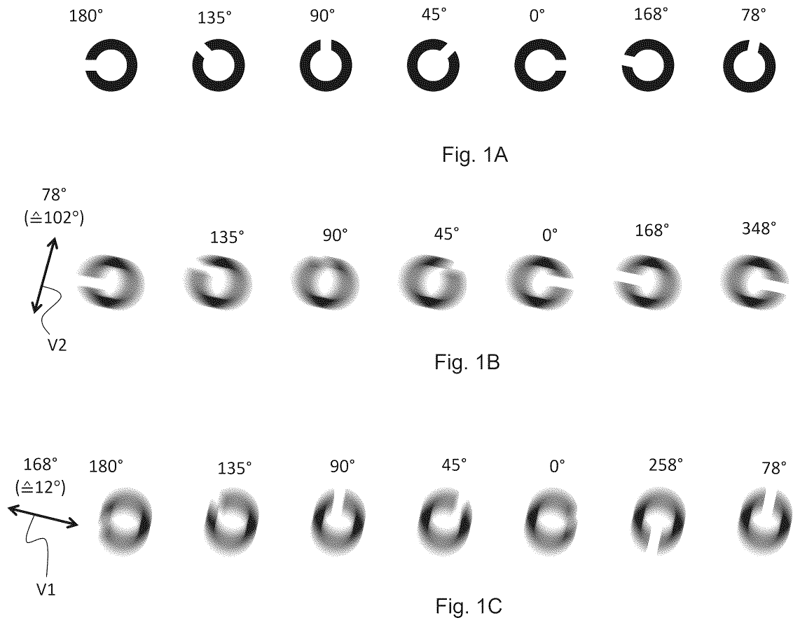

FIG. 1A shows exemplary embodiments of visual signs which may be displayed to the test person to determine his/her visual acuity. As visual signs, Landolt rings are used whose gap is aligned from left to right at the angles of 180°, 135°, 90°, 45° and 0°, respectively. These five left Landolt rings are typical visual signs which may also be used in conventional visual acuity determination.

On the right in FIG. 1A, two special and adapted visual signs are also shown, in which the gap is displayed aligned to the angles of 168° and 78°.

When determining his/her visual acuity as acuity-of-vision characteristics, an optical correction is applied and/or held up to the test person as an optical power through which the test person views the visual signs and can try to recognize them. For this purpose, a refraction unit may be used which is arranged, e.g., in front of the test person's eye(s).

Here, e.g., a refraction unit may be used by means of which only optical spherical corrections are (or can be) applied to the test person, but not necessarily also optical cylinder corrections. A refraction unit which only corrects spherically may thus be used, or a refraction unit with which only a limited selection of cylinder axes and/or optical cylinder corrections can be applied.

In order to determine the acuity-of-vision characteristics of the test person, a preferential direction is first selected from the visual defect data. The preferential direction is a direction in a plane which is arranged approximately perpendicular to the gaze direction of the test person. The preferential direction may be arranged in the same plane in which the cylinder axis of the cylindrical visual defect of the test person is defined.

Either the cylinder axis a and thus the first principal meridian of the cylindrical visual defect of the test person, or a direction perpendicular thereto in the same plane, α+90° and thus the second principal meridian of the cylindrical visual defect of the test person, may now be selected as the preferential direction. In the example, this would be a direction of 12° in a plane approximately perpendicular to the gaze direction of the test person as a first preferential direction for the first principal meridian and a direction of 102° in a plane approximately perpendicular to the gaze direction of the test person as a second preferential direction for the second principal meridian.

If the first preferential direction is selected, an optical spherical correction of s may be applied to the test person by means of the refraction unit, in the example +2.75 dpt. As a result, his/her visual defect is correctly corrected in the first preferential direction, but not in the other directions, in particular not perpendicular to the first preferential direction.

If the second preferential direction is selected, an optical spherical correction of s+z may be applied to the test person by means of the refraction unit, in the example −0.25 dpt (calculated from: +2.75 dpt-3.0 dps). As a result, his/her visual defect is correctly corrected in the second preferential direction, but not in the other directions, in particular not perpendicular to the second preferential direction.

If, e.g., an optical spherical correction of −0.25 dpt is thus applied to the test person, his/her visual defect is corrected relatively accurately at 102° in the second preferential direction V2. This correction is shown schematically in FIG. 1B on the far left. Since the cylinder axis of the visual defect is usually ascertained by looking at the test person's eyes, and the visual signs are usually displayed from the test person's gaze, the measurement angle of the cylinder axis is exactly mirror-inverted to the display angle of the visual signs. This means that the second preferential direction V2 is aligned at the display angle of 78° on a display on which the visual signs are displayed, which corresponds to a cylinder axis at the measurement angle of 102° looking at the test person's eyes.

For example, a display angle directed vertically upwards (corresponding to “12 o'clock”) corresponds to the 90° position. Likewise, a measurement angle directed vertically upwards (corresponding to “12 o'clock”) on the display plane corresponds to 90°.

A display angle directed to the right at “3 o'clock” corresponds to 0°. However, this display angle directed to the right corresponds to a measurement angle turned to the left (since it is mirror-inverted) looking at the display plane, i.e. a measurement angle of 180°.

This results in different angle values between the display angles which are defined on the display plane of the display and the measurement angles measured looking at the test person's eyes.

FIG. 1B shows the visual impression of the test person when looking at the visual signs shown in FIG. 1A. Here, the visual signs appear blurred, in particular in the direction perpendicular to the second preferential direction V2. The visual impression shown in FIG. 1B is ascertained by being calculated for the test person from the example with the visual defect data {s=+2.75 dpt; z=−3.0 dpt; α=12°}.

As shown in FIG. 1B, in particular the visual signs in which the gap is displayed at the display angles of 90° and 45° appear very blurred to the test person.

Specially adapted visual signs are now used for the test person, in which the gap is aligned perpendicular to the second preferential direction V2, i.e. at the display angles of 168° and 348°. The adapted Landolt ring at the display angle of 168° is also shown above in FIG. 1A as an adapted visual sign actually displayed. In these two adapted visual signs, a directional feature of the Landolt ring is aligned exactly in parallel to the second preferential direction V2, namely the transition from the black circular edge to the white gap and back to the black circular edge. Therefore, at least the gap of the two adapted Landolt rings “rotated” to the display angles of 168° and 348° appears to be relatively sharp to the test person, cf. the two visual impressions on the right in FIG. 1B. This is because the visual defect of the test person in his/her second principal meridian, i.e. along the second preferential direction V2, is fairly well and/or optimally corrected by holding up the optical spherical correction of −0.25 dpt.

The directional feature of the Landolt rings whose gap is displayed at the display angles of 90° and 45°, is relatively steep (i.e. almost perpendicular) to the corrected second preferential direction V2, which is why a very blurred visual impression results for these two visual signs in particular. However, a blurred visual impression also results for the other standard visual signs, making it impossible to determine visual acuity accurately.

If an optical spherical correction of +2.75 dpt is applied to the test person, his/her visual defect is corrected relatively accurately or optimally in the first preferential direction V1 at a measurement angle of 12°. This correction is indicated on the far left in FIG. 1C. This cylinder axis at the measurement angle of 12° appears on the display in the display plane at the display angle of 168°. Thus, the first preferential direction V1 is aligned on the display at the display angle of 168°.

FIG. 1C shows the visual impression of the test person when looking at the visual signs shown in FIG. 1A. Here, the visual signs appear blurred, in particular in the direction perpendicular to the first preferential direction V1. The visual impression shown in FIG. 1C is again ascertained by being calculated for the test person from the example with the visual defect data {s=+2.75 dpt; z=−3.0 dpt; α=12°}.

As shown in FIG. 1C, in particular the standard visual signs in which the gap is displayed at the display angles of 180°, 135° and 0° appear very blurred to the test person.

Specially adapted visual signs may now be used again for the test person, in which the gap is aligned perpendicular to the first preferential direction V1, i.e. at the display angles of 258° and 78°. The adapted Landolt ring at the display angle of 78° is also shown above in FIG. 1A as an adapted visual sign actually displayed. In the two adapted visual signs, the directional feature of the Landolt rings is aligned exactly in parallel to the first preferential direction V1, namely the transition from the black circular edge to the white gap and back to the black circular edge. Therefore, at least the gap of the two adapted Landolt rings rotated to the display angles of 258° and 78° appears to be relatively sharp to the test person, cf. the two visual impressions on the right in FIG. 1C. This is also because the visual defect of the test person in his/her first principal meridian, i.e. along the first preferential direction V1, is fairly well and/or optimally corrected by holding up the optical spherical correction of +2.75 dpt.

The directional feature of the Landolt rings whose gap is displayed at the display angles of 180°, 135° and 0° is relatively steep (i.e. almost perpendicular) to the corrected first preferential direction V1, which is why a very blurred visual impression results for these three visual signs in particular.

As part of a visual task for determining visual acuity, the test person corrected in his/her second preferential direction V2 with his/her optical spherical correction of −0.25 dpt may now be asked where the gaps of the two visual signs displayed at the display angles of 168° and 348° point, e.g. whether they point more to the left or right.

Alternatively or additionally, the test person corrected in his/her first preferential direction V1 with his/her optical spherical correction of +2.75 dpt may be asked, as part of another visual task for determining visual acuity, where the gaps of the two visual signs displayed at the display angles of 258° and 78° point, e.g. whether they point more upwards or downwards.

In this way, it can be checked whether the test person can still recognize the directional feature as a detail of the adapted visual sign or not. The visual acuity for the selected preferential direction V1 and/or V2 may be determined depending on the level of detail which the test person can still just barely recognize.

For the calculation of the resulting visual impression, which is shown in FIGS. 1B and 1C as well as in the following figures, it was assumed that the pupil diameter of the test person is 3.0 mm, the wavelength is 550 nm and the distance to the display is 5 meters. Furthermore, it was assumed that the visual signs are displayed as a rendered image with 1024×1024 pixels, wherein the image has a side length of 204.8 mm and the side length of a pixel corresponds to 40 μrad=0.1375 arcminutes.

The Snellen E also belongs to the same category of adapted visual signs as the Landolt rings. Here, the relevant feature, i.e. the directional feature of the Snellen E, is the sequence of:
dark area of an outer horizontal line,
light area of the background,
dark area of the central horizontal line,
light area of the background, and
dark area of the other outer horizontal line.

If necessary, the light background above and/or below may also form part of the sequence. Thus, when using Snellen Es, the longitudinal line connecting the horizontal lines must be oriented in parallel to the selected and corrected preferential direction V1 or V2, which only allows two different orientations, as with the Landolt ring.

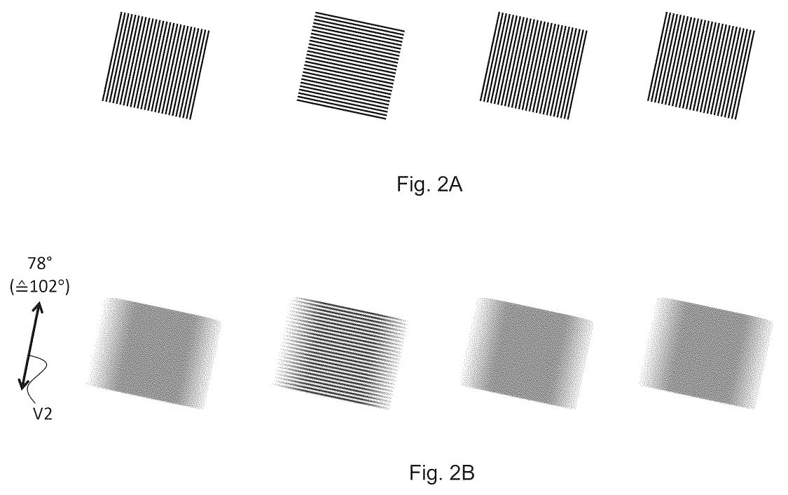

FIG. 2A shows further adapted visual signs for the test person with the visual defect used as an example. As adapted visual signs, squares hatched with continuous lines and without borders are used, which are displayed on the display. Here, the hatching lines of the first, third and fourth visual signs from the left are aligned in parallel to a display angle of 78°, while the hatching lines of the second visual sign from the left are aligned in parallel to a display angle of 168°.

The hatching lines of each visual sign all have the same thickness and the same alignment. In each case, two adjacent hatching lines always have the same and constant distance from each other.

The hatching lines provide a directional feature of the adapted visual signs. The direction of the directional feature is the direction of the alternation of the light and dark areas, i.e. the direction perpendicular to the hatching lines.

If the test person is in turn corrected for his/her second preferential direction V2 by applying a purely spherical optical correction of −0.25 dpt, the visual impression shown in FIG. 2B results for the test person. The first, third and fourth visual signs from the left appear as grey spots, while the hatching of the second visual sign can be recognized by the test person.

Thus, as part of a visual task for determining visual acuity, the test person can recognize the adapted visual sign which differs from the others. In the example, it is the second visual sign from the left whose directional feature is aligned in parallel to the selected and corrected second preferential direction V2, i.e. the display angle of 78° corresponding to the measurement angle of 102°, i.e. the second principal meridian of the cylindrical visual defect of the test person.

Here, the distance between two adjacent hatching lines and/or the thickness of the black hatching lines may be used as the size of the recognized detail for determining the visual acuity.

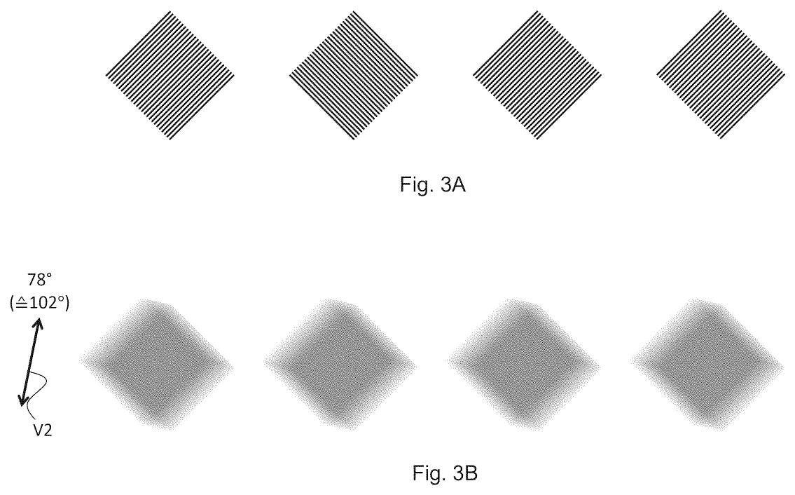

FIG. 3A shows further visual signs for the test person with the visual defect used as an example. As visual signs, squares hatched with continuous lines and without borders are again used, which are displayed on the display. Here, the hatching lines of the first, third and fourth visual signs from the left are aligned in parallel to a display angle of 315°, while the hatching lines of the second visual sign from the left are aligned in parallel to a display angle of 135°.

Here, the size of the squares and the distance and thickness of the hatching lines may correspond to the visual signs shown in FIG. 2A. Again, the second visual sign from the left is different from the others.

If the test person is in turn corrected for his/her second preferential direction V2 by applying a purely spherical optical correction of −0.25 dpt, the visual impression shown in FIG. 3B results for the test person. Since none of the visual signs is well adapted to the corrected second preferential direction V2 at the display angle of 78°, the test person cannot recognize a single hatching of the visual signs because the visual impression of a grey spot or blurred square results for him/her in each of the four visual signs.

The visual impression thus differs significantly from the visual impression which results from the visual signs optimized for the test person, cf. FIGS. 2A and 2B.

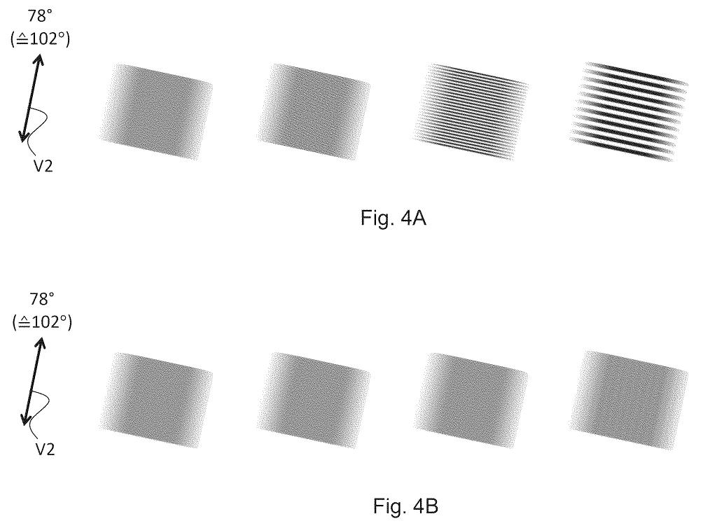

FIG. 4A shows the visual impression of the test person when corrected by applying a purely spherical optical correction of −0.25 dpt for his/her second preferential direction V2 at the display angle of 78°. Here, four borderless hatched squares are displayed as visual signs, the hatching lines of which are displayed in parallel to the display angle of 168°. Thus, the directional feature of these visual signs is aligned in parallel to the corrected second preferential direction V2, and the test person can make out at least the hatching lines of some of the adapted visual signs, e.g. the two right-hand adapted visual signs.

However, the hatching lines of the visual signs have different widths and different distances. The left visual sign has a distance between two black hatching lines of log MAR −0.66, the second visual sign from the left of −0.26, the third visual sign from the left of 0.14 and the fourth visual sign from the left of 0.54. This distance may be used as a measure of visual acuity. Thus, if the test person only recognizes the two right visual signs, of which the finer shaded visual sign (i.e. the third from the left) has a distance of log MAR 0.14, this smallest detail still recognized may be used for visual acuity determination.

FIG. 4B shows the visual impression of the test person when he/she has been corrected in turn by applying a purely spherical optical correction of −0.25 dpt for his/her second preferential direction V2 at the display angle of 78°. Again, four borderless hatched squares are displayed here as visual signs, the hatching lines of which have different widths and different distances as in FIG. 4A. From left to right, the visual signs have a distance between two adjacent black hatching lines of log MAR −0.66; −0.26; 0.14 and 0.54, just as in FIG. 4A.

The hatching lines are displayed in parallel to the display angle of 78°, which is why they appear as blurred as possible to the test person; this is because the directional features of the visual signs are aligned perpendicular to the corrected second preferential direction V2 here. Thus, the visual signs used in FIG. 4B can at most be used as visual signs appearing grey, but not as adapted visual signs recognizable by the test person thus corrected.

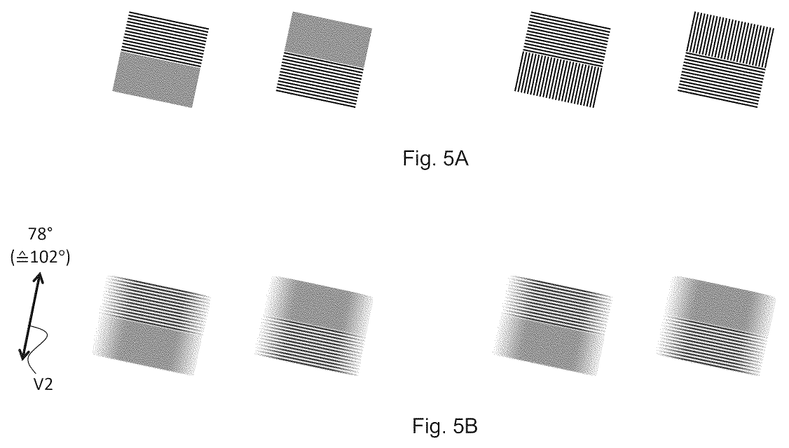

FIG. 5A shows further visual signs for the test person with the visual defect used as an example. As visual signs, squares hatched with continuous lines and without borders are used, which are displayed on the display. Here, the hatching lines of all the visual signs are aligned in parallel to a display angle of 168°. However, only half of the squares are provided with said hatching lines here. In the first and third visual signs from the left, the upper halves are provided with the hatching, respectively, while in second and fourth visual signs from the left, the lower halves are provided with the hatching. The other halves are each filled gray in the first and second visual signs from the left, and in the third and fourth visual signs from the left with a hatching perpendicular thereto, i.e. aligned in parallel to a display angle of 78° (shortened).

The hatching lines provide a directional feature of the adapted visual signs. The direction of the directional feature is the direction of the alternation of the light and dark areas, i.e. the direction perpendicular to the hatching lines.

If the test person is in turn corrected for his/her second preferential direction V2 by applying a purely spherical optical correction of −0.25 dpt, the visual impression shown in FIG. 5B results for the test person. In the first and third visual signs from the left, the hatching appears in the upper half, and in the other two, in the lower half.

The other half of visual sign each appears as a gray spot. It makes hardly any difference to the test person whether the other half is actually filled with a medium gray value or with the hatching lines parallel to the selected second preferential direction.

Thus, as part of a visual task for determining visual acuity, the test person may be asked to distinguish between the displayed adapted visual signs depending on his/her visual acuity.

Here, visual signs which have differently filled areas, in particular a hatching which only fills part of the adapted visual sign, may also be used.

Here, the distance between two adjacent hatching lines and/or the thickness of the black hatching lines may again be used as the size of the recognized detail for determining the visual acuity.

As an alternative to the hatched squares shown in the figures, all types of figures such as circles, rectangles, symbols, animals, letters, etc. which are filled with hatching may be used as adapted visual signs. Preferably, they do not have any border lines which could influence the visual impression of the hatching.

Here, the hatching lines may be perpendicular to the selected and corrected preferential direction V1 or V2, since the directional feature and relevant feature is the alternation of light and dark areas of the hatching lines. Here, it is favorable for the method if the figure itself has as few details as possible, such as e.g. a circle or a square. Here, in order to adapt the alignment, the entire visual sign including the hatching or only the hatching within the visual sign may be rotated. The hatching may be designed to be binary, i.e. with hard black edges, or continuous, e.g. with a sinusoidal intensity course.

### Visual Tasks

In order to determine visual acuity, the test person may be given visual tasks in which adapted visual signs are displayed. In the visual tasks, a distinction may be made between visual tasks with active and passive feedback of the test person. Here, active feedback may be understood as a statement made by the test person, e.g. either verbally or by consciously looking at a visual sign and acquiring the gaze direction by means of eye tracking. Passive feedback may be understood as the tracking of a presented visual sign that is moving. Here, it may be concluded whether the visual sign is still reliably recognized based on an eye movement acquired by means of an eye tracking unit.

The visual signs are displayed with a defined presentation type and thus presented to the test person. Here, a presentation type is understood to mean properties such as contrast, size or frequency of the hatching. Here, size is a particularly important property for visual signs of the type which can be displayed in two ways that are mirror-inverted with respect to the selected preferential direction, e.g. Landolt rings and Snellen Es. The frequency of the hatching is a particularly important property for visual signs of the type which have hatched areas and/or consist of hatched areas.

The presentation type may be degraded by changing the presentation type to poorer recognizability, e.g. reducing the size (in particular in the case of adapted visual signs of the type which can be displayed in two ways that are mirror-inverted with respect to the selected preferential direction), reducing the contrast and/or increasing the frequency of the hatching (in particular in the case of adapted visual signs of the type which have hatched areas and/or consist of hatched areas).

In one embodiment of a visual task with active feedback, one or more adapted visual signs of the type which can be displayed in two ways that are mirror-inverted with respect to the selected preferential direction. In these displayed visual signs, the orientation of the visual signs is to be recognized by the test person.

Displaying several visual signs of the same presentation type allows a more reliable assessment of the response. Here, a degradation in the presentation type up to the point at which the visual signs can no longer be recognized allows to determine the visual acuity.

In one embodiment of a visual task with active feedback, one or more adapted visual signs of the type which have hatched areas and/or consist of hatched areas are presented. The test person is asked to recognize the presence of a hatching of the visual sign.

Displaying several visual signs of the same presentation type allows a more reliable assessment of the response. A degradation in the presentation type up to the point at which the visual signs or the presence of a hatching can no longer be recognized allows to determine the visual acuity.

Furthermore, one or more adapted visual signs with adapted orientation as well as one or more adapted visual signs with an orientation deviating therefrom may also be presented, e.g. with an orthogonal orientation. In the case of visual signs of the type which have hatched areas and/or consist of hatched areas, the visual signs may be presented with uniform filling and the test person may be asked whether he/she can recognize differences and/or which one or more visual signs deviate from the others. This type of visual task is also referred to as a “forced choice”.

In one embodiment of a visual task with passive feedback, one or more adapted visual signs of one of the aforementioned types are presented which is moving. Here, the presentation type may be degraded continuously and/or gradually. Based on the eye movement acquired by means of an eye tracking unit, the presentation conditions under which the visual sign and/or visual object is still reliably recognized may be concluded. The visual acuity of the test person may be derived and/or determined therefrom.

The visual signs may be displayed by means of a light field display. Here, the applied optical spherical corrections do not have to be physically applied but may be simulated as wavefronts.

Combination with Eye Tracking

In visual tasks with active feedback, eye tracking allows the operation to be automated, in visual tasks with passive feedback, eye tracking may be absolutely necessary.

In one embodiment, the gaze direction of the test person may be determined when solving the visual task by means of at least one image and/or video of the pupil and/or one or more Purkinje reflexes. The gaze direction may also be determined from a combination, e.g. by means of Purkinje reflexes ascertained in a video of the pupil. For this purpose, the apparatus for determining the visual acuity may have at least one calibrated image capturing device, e.g. a digital camera.

In one embodiment, an eye tracking unit is used to position an optical unit, i.e. for centering and/or focusing the optical unit. For example, a measuring head of the device may be centered and/or focused. This positioning may then be kept constant and this eye tracking unit may be used to determine the gaze direction of the test person, e.g. for passive and/or active response to a visual task. Here, different tasks may be solved with just one single eye tracking unit.

Integration into an Optometric Measurement Device

The determination of visual acuity may be combined with a determination of visual defect, in particular to determine visual defect according to sphere, cylinder and axis. Here, lower and possibly higher order aberrations may also be ascertained.

For this purpose, an apparatus for determining visual acuity is connected and/or combined with an autorefraction or aberrometry unit.

In one embodiment, an autorefractometer and/or an aberrometer is assumed here as the refraction unit, which has a display unit and an optical unit which is used to show the adapted visual sign and thus the target. If necessary, this unit may also be used for fogging to determine the visual acuity without the need for additional optical components.

Ideally, the display unit is designed here as a programmable display to be able to display adapted representations for the various tasks. Alternatively, the visual sign display may also be used as being fogged for the autorefractometric and/or aberrometric measurement. By means of a beam splitter and/or mechanical means, it is possible to switch between the displays.

Such an apparatus allows to first determine the visual defect with the aid of the autorefractometer and/or the aberrometer, and to derive the visual defect data from the result of this measurement and, therefrom, the preferential direction with the power to be applied for this preferential direction.

In such an apparatus, an existing eye tracking unit used for centering and/or focusing the autorefractometric and/or aberrometric measurement may be used for eye tracking of the gaze direction of the test person.

In the apparatus, the same unit may be used to present the at least one visual sign and thus the target and, if necessary, a fogging as part of the autorefractometric and/or aberrometric measurement. For autorefractometric and/or aberrometric measurements, the use of a comparatively small visual sign as a target may be sufficient or even useful, since it already preadjusts the test person's gaze before fine adjustment is effected by looking at an excellent visual sign.

In the fogged state, a small visual sign may be used as a target and/or be advantageous, since—if the test person does not recognize any details—the small bright spot of a small target controls the gaze better than a more extended one.

In contrast, a larger visual sign is often useful as a target for determining visual acuity, since it allows the presentation of several different visual signs and, in particular in visual tasks in which a gaze movement is acquired, allows a larger gaze movement. This can be achieved with a sub-device which allows both the vergence and/or divergence, i.e. the “optical distance”, of the light of the presented target and the size of the image of the presented target. This can be achieved, for example, by combining a display with two axially movable spherical lenses in which the axial distances of both lenses can be adjusted independently of the display.

One or more other measurement units may be integrated in addition or instead, such as an opacity unit, a topography and/or topometry unit, a Scheimpflug camera and/or a tonometry unit. Here as well, individual components of several units may be used.

### Influence of Accommodation and Other Principal Meridian

If the precise design of the visual signs and/or the visual task can lead to an influence of accommodation and/or the non-observed principal meridian, the method may be varied as follows.

Due to accommodation, the planes in which both principal meridians are in focus are pulled forward (i.e. in the direction from the retina to the eye lens). In this embodiment, therefore, the posterior principal meridian (i.e. one that is less refracted by the eye) is imaged sharply on the retina with the spherical correction and/or, if fogging is desired (e.g. as part of a sensitivity determination), is imaged in front of the retina. In order to avoid an influence of accommodation, the direction in which the principal meridian less refracted by the eye is in focus is selected as the preferential direction for the presentation of the visual signs.

According to one embodiment, the principal meridian for which there is weaker refraction in the test person's eye is thus selected as the preferential direction. This can reduce the impact of accommodation on the visual acuity determination.

In order to avoid the influence of the other principal meridian when determining the visual acuity for an optical spherical correction deviating from the best correction in the principal meridian being observed, e.g. in the case of an applied blur, e.g. to determine a sensitivity, the correction may deviate from the direction which would be required for the correction in the other principal meridian.

Here, in particular with regard to the above consideration of accommodation, an observation of the less refractive principal meridian and a blur in the positive direction is used, which is also referred to as fogging in the narrower sense.

Thus, in one embodiment, the principal meridian for which a weaker refraction occurs in the test person's eye is selected as the preferential direction. In order to determine sensitivity, determination of a visual acuity value is performed by applying an optical spherical correction which deviates in a positive direction from the ascertained optimal optical spherical correction. In addition, a visual acuity value may be determined by applying the ascertained optimal optical spherical correction. From these two visual acuity values, both the visual acuity and sensitivity of the test person can be ascertained as accurately as possible, wherein both the influence of accommodation and the other principal meridian can be reduced.

Taking into Account HOA

If a wavefront measurement of the eye is performed, possibly also taking into account higher order aberrations (abbreviated as HOA), this wavefront measurement may be used instead of the objectively and/or subjectively ascertained refraction values to provide the visual defect data therefrom. Thus, the preferential direction may be selected based on the wavefront measurement to which the adapted visual signs are oriented. Furthermore, the strength of the optical spherical correction to be applied for this selected preferential direction may also be derived from the wavefront measurement.

Here, a point spread function may be determined from the wavefront data, the optical spherical correction (and/or the spherical corrections) applied when determining the visual acuity and a pupil size according to methods known from the literature. Instead of determining the preferential direction of the adapted visual signs based on the subjective and/or objective refraction in the second (Zernike) order, the preferential direction may be derived from the point spread function, e.g. from the direction and/or axis of the smallest extension of the point spread function. Here, the direction of least confusion may be selected, e.g. as the direction of the smallest standard deviation of the point spread function.

### Determining Sensitivity

In some embodiments, the sensitivity of at least one eye of a test person or spectacle wearer is ascertained. This allows the calculation, optimization or evaluation of a spectacle lens for the at least one eye of the test person to be performed, taking into account the ascertained sensitivity of the at least one eye of the test person. This may be used in manufacturing a spectacle lens.

In methods for optimizing a spectacle lens according to the prior art, a spectacle lens is optimized by minimizing or maximizing a target function in which actual values and corresponding target values of at least one imaging property or aberration of the spectacle lens are included. The at least one imaging property or aberration may represent a direct quantification of a wavefront deviation from a reference wavefront. An exemplary target function is, e.g., the function:

$$F = {\sum }_{i} [ {{G}_{R , i} ( {R}_{a c t ual} ( i ) - {R}_{target} ( i ) )}^{2} + {{G}_{A , i} ( {A}_{a c t u a l} ( i ) - {A}_{SPK , target} ( i ) )}^{2} + … ] ,$$

where:
   i (i=1 to N) denotes an evaluation point of the spectacle lens;
   R_{actual}(i) denotes the actual spherical power or the refractive error at the i^{th} evaluation point;
R_{actual}(i) denotes the target spherical power or the target refractive error at the i^{th} evaluation point;
Ast_{actual}(i) denotes the astigmatism or the astigmatic error at the i^{th} evaluation point;
Ast_{target}(i) denotes the target astigmatism or the target astigmatic error at the i^{th} evaluation point.

The variables G_{R,i}, G_{A,i} are weights of the respective imaging property or aberration which are used in the optimization.

Direct quantification of a wavefront deviation in diopters without taking into account the effective pupil size is not the best possible criterion to describe and assess the perception of a spectacle wearer through a spectacle lens due to the depth of field depending thereon. On the basis of this finding, DE 10 2017 007 663 A1 proposes to directly take into account the visual acuity (acuity of vision) in the target or quality function. The visual acuity included in the target or quality function depends, via an assignment, on at least one imaging property or aberration of a spectacle lens system, wherein the at least one imaging property or aberration may be evaluated on a suitable evaluation surface (e.g. on the vertex sphere or in the eye). The spectacle lens system may consist of at least one spectacle lens (e.g. a spectacle lens of refractive spectacles). Preferably, however, the spectacle lens system comprises further components such as a model eye or eye model which may be based on average values of spectacle wearers or on at least one individual parameter of the eye of the spectacle wearer. Stated differently, the spectacle lens system which is based on the assignment of at least one imaging property or aberration to the visual acuity of the spectacle wearer may be a spectacle lens-eye system.

As described in DE 10 2017 007 663 A1, an exemplary target or quality function, which depends on the visual acuity V via the assignment of the at least one imaging property or aberration ΔU_{s,j} to the visual acuity of the spectacle wearer or an average spectacle wearer, may have, e.g., the following structure:

$${F}_{s} = {\sum }_{i} [ {{G}_{s , j , i}^{V} ( {V}_{a c t u a l} ( Δ {U}_{s , j} ( i ) ) - {V}_{t a r g e t} ( Δ {U}_{s , j} ( i ) ) )}^{2} + … ] .$$

In the above formula, V(ΔU_{s,j}(i)) denotes a function which describes the dependence of the visual acuity on at least one imaging property or aberration of a spectacle lens system at the i^{th} evaluation point (i=1, 2, 3, . . . , N) on an evaluation surface. Stated differently, V(ΔU_{s,j}(i)) describes an exemplary assignment of at least one imaging property or aberration of a spectacle lens system to the visual acuity of the test person or spectacle wearer or an average spectacle wearer when viewing an object through the spectacle lens system. The argument ΔU_{s,j} is generic and may denote any imaging property or aberration of a spectacle lens system which describes the effect of the spectacle lens system on a light beam emanating from an object or the difference of the effects of the spectacle lens system on a light beam emanating from an object and on a reference light beam converging on the retina of the eye. Here, one or more image property(ies) or aberration(s) may be included in the target or quality function and evaluated, wherein the subscript j,j≥1 denotes the j^{th} image property or aberration.

V_{actual}(ΔU_{s,j}(i)) denotes the visual acuity which is ascertained based on the assignment and the actual value of the at least one imaging property of the spectacle lens to be calculated (e.g. to be optimized) or evaluated at the i^{th} evaluation point, and V_{target}(ΔU_{s,j}(i)) denotes the corresponding target value of the visual acuity.

The at least one imaging property or aberration may be calculated or evaluated on a suitable evaluation surface. Accordingly, the subscript “s” stands for any evaluation surface of the at least one imaging property or aberration ΔU_{s,j}. The evaluation surface may, e.g., be a plane (evaluation plane) or a curved (e.g. spherical) surface. The evaluation surface may be, e.g., the vertex sphere or a surface in the eye, e.g. one of the following planes or surfaces:
a plane or a (e.g. spherical) surface behind the cornea,
the anterior surface of the crystalline lens or a plane tangential to the anterior surface of the crystalline lens,
the posterior surface of the crystalline lens or a plane tangential to the posterior surface of the crystalline lens,
the plane of the exit pupil (EP); or
the plane of the lens posterior surface (L2).

The variable

$${G}_{s , iso , i}^{V}$$

denotes the weighting of the visual acuity specified by the assignment to the imaging property ΔU_{s,j} at the i^{th} evaluation point.

Here, e.g., one of the visual acuity models described in DE 10 2017 007 663 A1 or any other suitable visual acuity model (which in particular describes the visual acuity as a function of the refraction or refraction deficit) may be used, preferably in combination with a specification of how the visual acuity model is to be incorporated into the target function of an optimization in conjunction with a transformation of the target specifications and weights. At this point, it is noted that, in the context of this description, a sensitivity metric (as described below) may preferably be used based on such a visual acuity model (as a functional dependence of a visual acuity value on the refraction/refraction deficit). In particular, a preferred sensitivity metric could be used as a derivative of a visual acuity model (i.e. the function of the visual acuity value on the refraction/refraction deficit) according to the refraction/refraction deficit.

With the aid of the target function, evaluation of a spectacle lens can also be performed, wherein the actual value of the at least one imaging property of the spectacle lens to be evaluated is calculated at at least one evaluation point of the spectacle lens to be evaluated and compared with the corresponding target value.

As can also be seen from DE 10 2017 007 663 A1, knowledge of the so-called sensitivity, i.e. the change in visual acuity with the refraction deficit, is especially helpful for calculating, optimizing and/or manufacturing highly individual and high-quality spectacle lenses. Thus, the assignment of at least one imaging property or aberration of a spectacle lens system to the visual acuity of the spectacle wearer or the function V(ΔU_{s,j}(i)) may depend parametrically on the measured initial visual acuity and/or the ascertained sensitivity of the spectacle wearer.

Sensitivity is a (in particular phenomenological) quantity or parameter used in spectacle optics and ophthalmology, with which the dependence of visual acuity on a refraction deficit can be described or indicated. The sensitivity of an eye is understood in particular as the change in the visual acuity of the eye in the event of a change in a refraction deficit. In particular, the sensitivity may be defined as the derivative of the visual acuity after the refraction deficit or as the local derivative of the visual acuity after the refraction deficit at a certain refraction deficit. Here, the refraction deficit is a deviation of a power or refraction held up to at least one eye of the test person during the visual acuity determination from an ideal refraction ascertained or known for the at least one eye. The ideal refraction (hereinafter also referred to as optimal refraction or target refraction) may be ascertained, e.g., from a conventional objective and/or subjective refraction measurement. In particular, the sensitivity describes how much the visual acuity changes when an optical power or correction in front of the eye changes. The sensitivity may be described quantitatively in particular with the aid of a sensitivity metric and/or with the aid of a visual acuity model.

The sensitivity of the at least one eye of a test person can thus be taken into account when calculating and/or creating individual spectacle lenses, in particular when creating multifocal spectacle lenses such as ophthalmic spectacle lenses. Spectacle lenses may have transitions between regions with different optical corrections, e.g. transitions between a far viewing point and a near viewing point. Precisely these transitions between spectacle lens regions with different optical corrections may be designed differently. They are referred to as hard transitions or soft transitions, for example, depending on how strong or gentle the change in refraction is along the transition. In the case of highly individualized and high-quality spectacle lenses, such a transition (but other regions of the spectacle lens) may be adjusted to the sensitivity of the at least one eye of the test person or spectacle wearer.

In order to determine the sensitivity of the at least one eye of a test person with regard to blur, at least two applied powers and the visual acuity achieved therewith in each case are required. Within the scope of the invention, these may be ascertained as visual acuity-refraction value pairs of the acuity-of-vision characteristics. Associated models and corresponding formulas for calculating the sensitivity are described below. According to the prior art, quantized powers are held up to the test person or to the at least one eye of the test person to ascertain the sensitivity (e.g. in steps of 0.25 dpt using conventional trial lens sets). Based on a visual acuity chart with visual signs in quantized sizes or quantized visual acuity levels, the corresponding visual acuity is ascertained for each of the powers held up. Furthermore, the optimal correction (or the optimal refraction or target refraction) must be ascertained for the test person in order to be able to convert the powers held up into a refraction deficit.

The double quantization associated with the conventional method leads to high measurement uncertainty. Conventional methods are not only time-consuming, but may also be psychologically unfavorable, since the at least one eye of the test person is provided with a poorer correction after determining the optimal refraction and the test person must then solve visual tasks with this poorer correction to ascertain the sensitivity. This order is necessary in the conventional procedure, since a defined fogging for visual acuity measurement can only be set once the optimal refraction is known.

According to one aspect, it is therefore an object of the present invention to ascertain the sensitivity of the at least one eye of the test person, required in particular for the calculation, optimization, evaluation and/or manufacture of highly individualized and high-quality spectacle lenses, in an improved manner, in particular simply and quickly. Furthermore, it may be an object of the present invention to provide a method and an apparatus for calculating, optimizing, evaluating and manufacturing spectacle lenses which are highly individual and of high quality due to the consideration of the sensitivity of the at least one eye of the test person. It may also be an object of the present invention to provide such improved spectacle lenses.

### Determining the Sensitivity as Acuity-of-Vision Characteristics by Varying the Applied Optical Power

In some embodiments, the sensitivity of at least one eye of a test person is determined as acuity-of-vision characteristics on the basis of at least two visual acuity-refraction value pairs provided.

While according to a first alternative, the applied optical power is kept constant and changed, in a second alternative, the dimension of the directional feature of the adapted visual sign is kept constant and the applied optical power is varied. This second alternative is explained in more detail below.

In this second alternative, the visual acuity-refraction value pairs may be provided by the following steps:
projecting a target, which may contain at least one adapted visual sign, with an adjustable target refraction corresponding to the applied optical power into the at least one eye of the test person, wherein the target is designed to verify a specified visual acuity; and
ascertaining a visual acuity limit refraction of the at least one eye of the test person associated with the specified visual acuity by varying the target refraction of the target projected into the at least one eye of the test person and acquiring a test person action by which it is detected that the identifiability of the target for the test person has changed at the time of the test person action.

As already mentioned above, the “sensitivity” (with regard to blur) of at least one eye of the test person is understood to mean the dependence of the visual acuity of the at least one eye of the test person on a refraction deficit, wherein the “refraction deficit” is a deviation of a power or refraction held up to the at least one eye of the test person during the visual acuity determination from an ideal or optimal refraction (target refraction) ascertained or known for the at least one eye.

The “visual acuity” is a measure of the (central) acuity of vision of the at least one eye of a test person. Usually, the visual acuity is ascertained in bright light. In particular, visual acuity may be defined as the reciprocal of the smallest recognizable gap in the standard visual sign, the Landolt ring. In humans, visual acuity may be determined by means of an eye test. For this purpose, visual signs (“optotypes”) are presented to the test person and it can be seen from the test person's answers whether the test person has recognized them correctly. The visual acuity depends on which visual signs the test person can recognize with the set and/or applied refraction. The visual signs usually have a defined size, brightness, shape and a defined contrast. The visual signs may be displayed on a board or projected.

In the method according to the invention, the target comprises at least one adapted visual sign per visual task, in which the directional feature is arranged in parallel to the selected preferential direction.

The use of a projector instead of a board has the advantage of being independent of the test distance. DIN regulations exist for reproducible visual acuity testing. According to these, the standard visual sign is the so-called Landolt ring, a ring of defined width with a gap of the same width, which can be arranged in eight different directions. By recognizing the direction of the gap, the test person demonstrates that his/her resolution is at least equal to the width of the gap.

In practice, however, standardized images of numbers are usually used as visual signs because they are easier to understand. There are also other standardized visual signs, such as the “Snellen E”, the “Pflüger-E-hook”, in which the central line is shorter, as well as others that are suitable for testing the visual acuity of illiterate people and children of pre-school age, as well as for non-verbal communication.

When determining visual acuity, a distinction is made between that with correction, such as spectacles or contact lenses, and that without correction. Here, acuity of vision without correction is also referred to as raw visual acuity. The abbreviations “s.c.” (“sine correctione”, Latin for “without correction”) and “c.c.” (“cum correctione”, Latin for “with correction”) are also frequently used.

The sensitivity of the at least one eye may be ascertained in particular on the basis of a sensitivity metric. By using a sensitivity metric, a sensitivity may be calculated even when the applied refraction values do not have a specified distance from each other.

The sensitivity metric represents the dependence of the visual acuity on a refraction deficit. Here, the distance between two refraction values may be part of a sensitivity metric. The sensitivity metric may be defined in the metric space of the refraction values. Each refraction value of the sensitivity metric may be assigned a visual acuity value or vice versa. For example, the refraction may be defined in an at least three-dimensional space. In this way, a refraction value may usually be described using the coordinates s, c and α. Here, s may be dependent on the strength of an optical correction of the sphere, c on the strength of an optical correction for a cylinder, and α on the cylinder axis of this cylinder. The strength of the optical correction for the cylinder is sometimes referred to as z as an alternative to c. In this metric space of the refraction values, at least the refraction values for a specified first and a specified second visual acuity may be determined and are therefore known when calculating the sensitivity. The sensitivity metric may be used to determine the sensitivity depending on two different, basically arbitrary refraction values. By using such a sensitivity metric, the determination of the sensitivity is independent of visual acuity measurements at specified refraction values, as is usual with conventional methods. Here, on the one hand, the determination of the sensitivity may become independent of visual acuity measurements at at least one predetermined and/or fixedly specified refraction distance from the refraction result (or from the optimal refraction or target refraction), and on the other hand of visual acuity measurements at at least one predetermined and/or fixedly specified relative refraction distance between the two applied refractions. This can make it easier for both the refraction specialist and the test person to ascertain the measurement data required for sensitivity determination.

### Exemplary Embodiments of a Sensitivity Metric

The sensitivity may be calculated using a metric space in which different refraction values represent individual points. A refraction value may be represented in three dimensions, e.g. with the coordinates s, c, and α. Here, s may depend on the strength of a spherical correction and may, e.g., be given in diopters (which can also be abbreviated to dpt). c may depend on the strength of a cylinder correction and can be indicated in dpt, for example, α may depend on the cylinder axis of the cylinder correction and can be indicated in degrees, e.g. from 0 to 180°. Alternatively, other coordinates may be used.

In the following, it is assumed by way of example that the best refraction (also referred to as optimal or ideal refraction in the context of this description), i.e. in particular a specific objective, subjective refraction result, in this sensitivity metric is denoted by s₀, e₀, and g₀ and the associated visual acuity is denoted by v₀. When performing the method, at least two visual acuity-refraction value pairs are provided. In general, n refractions sᵢ, cᵢ, αᵢ with the associated visual acuity vᵢ with i∈[1, . . . , n] and n≥2 may be provided. Here, at least one visual acuity-refraction value pair of the at least one eye of the test person may already be known and provided as a known value pair. The provision comprises in particular an ascertainment and/or measurement.

In a possible sensitivity metric, the distance of a refraction i from the best refraction in the mean sphere di and in the cylinder a; is calculated using equation (1):

$$\begin{matrix}{d}_{i} = ( {s}_{i} + \frac{1}{2} \cdot {c}_{i} ) - ( {s}_{o} + \frac{1}{2} \cdot {c}_{0} ) \\ {α}_{i} = \sqrt{{c}_{i}^{2} + {c}_{0}^{2} - 2 \cdot {c}_{i} \cdot {c}_{0} \cdot cos ( {α}_{i} - {α}_{0} )}\end{matrix} \qquad ( 1 )$$

Simple Bilinear Model of a Sensitivity Metric with Knowledge of a Target Refraction

In one embodiment of a bilinear model of a sensitivity metric, for the dependence of the visual acuity, the following relationship shown in equation (2) applies for each individual measurement for a refraction i. Here, in a simplified case, it can be assumed that the test person cannot compensate for fogging by accommodation.

$$lg {v}_{i} = {m}_{d} \cdot \vert {d}_{i} \vert + {m}_{a} \cdot {a}_{i} + lg {v}_{0} \qquad ( 2 )$$

Here, m_{d} stands for the sensitivity at a spherical distance and mₐ for the sensitivity at a cylindrical distance. Such a separation between a spherical and a cylindrical refraction deficit may be used to take into account that test persons may react very differently to these two components of a refraction deficit. For example, from data from D. Methling: Determination of visual aids, 2nd ed. Ferdinand Enke Verlag, Stuttgart 1996, it can be ascertained that equations (3) apply to the population average as empirically ascertained:

$$\begin{matrix}{m}_{d} = \frac{2}{1 d p t} \cdot lg 0 , 5 = - 0 , 601 d p {t}^{- 1} \\ {m}_{a} = \frac{1}{1 d p t} \cdot lg 0 , 5 = - 0 , 301 d p {t}^{- 1}\end{matrix} \qquad ( 3 )$$

In general, the above equation (2) has the independent parameters mₐ, m_{d}, v₀. Therefore, the system of equations (2) with three measurements i=1, 2, 3 of refractions (s₁, c₁, α₁; s₂, c₂, α₂; s₃, c₃, α₃) at three (in particular specified) different visual acuity values v₁, v₂, v₃ can be solved uniquely to form the system of equations (2a):

$$\begin{matrix}{m}_{a} = - \frac{1}{deno min a t o r} ( log ( {v}_{1}^{\vert {d}_{2} \vert - \vert {d}_{3} \vert} ) + log ( {v}_{2}^{\vert {d}_{3} \vert - \vert {d}_{1} \vert} ) + log ( {v}_{3}^{\vert {d}_{1} \vert - \vert {d}_{2} \vert} ) ) \\ {m}_{d} = - \frac{1}{deno min a t o r} ( log ( {v}_{1}^{{a}_{3} - {a}_{2}} ) + log ( {v}_{2}^{{a}_{1} - {a}_{3}} ) + log ( {v}_{3}^{{a}_{2} - {a}_{1}} ) ) \\ log {v}_{0} = - \frac{1}{deno min a t o r} ( log ( {v}_{1}^{{a}_{2} \vert {d}_{3} \vert - {a}_{3} \vert {d}_{2} \vert} ) + log ( {v}_{2}^{{a}_{3} \vert {d}_{1} \vert - {a}_{1} \vert {d}_{3} \vert} ) + log ( {v}_{3}^{{a}_{1} \vert {d}_{2} \vert - {a}_{2} \vert {d}_{1} \vert} ) ) \\ with denominator = ( {a}_{2} - {a}_{3} ) \vert {d}_{1} \vert + ( {a}_{3} - {a}_{1} ) \vert {d}_{2} \vert + ( {a}_{1} - {a}_{2} ) \vert {d}_{3} \vert\end{matrix} \qquad ( 2 a )$$

In this case, e.g., a visual acuity measurement may take place at optimal correction conditions, i.e. at the target refraction (in particular ascertained from an objective and/or subjective refraction measurement). Then, e.g., the following applies for i=3: (s₃, c₃, α₃)=(s₀, c₀, α₀). At this optimal correction condition, a₃=a₀=0 and d₃=d₀=0. Thus, the third of the equations (2a) is automatically satisfied. The other equations then assume the following form of the system of equations (4):

$$\begin{matrix}{m}_{a} = - \frac{1}{deno min a t o r} ( \vert {d}_{2} \vert log ( {v}_{1} / {v}_{0} ) - \vert {d}_{1} \vert log ( {v}_{2} / {v}_{0} ) ) \\ {m}_{d} = - \frac{1}{deno min a t o r} ( - {a}_{2} log ( {v}_{1} / {v}_{0} ) + {a}_{1} log ( {v}_{2} / {v}_{0} ) ) \\ with denominator = {a}_{2} \vert {d}_{1} \vert - {a}_{1} \vert {d}_{2} \vert\end{matrix} \qquad ( 4 )$$

The system of equations (4) thus provides an exemplary embodiment of a simplified bilinear model of a sensitivity metric. The system of equations (4) can be solved with knowledge of the target refraction and with knowledge of two additional refraction values for two additional visual acuity values (for i=1,2). The visual acuity-refraction value pairs used here may be ascertained using the method according to the invention. Thus, the sensitivity can be ascertained from the system of equations (4). The sensitivity describes the dependence of the visual acuity on the refraction (deficit). This can be described, e.g., by the values mₐ and m_{d}.

If, in addition to the visual acuity v₀ at the target refraction, more than two additional refractions are measured at specified visual acuity values, the sensitivity can be ascertained more precisely by determining m_{d} and mₐ from all the data via an equalization method, e.g. the least squares method. Furthermore, outliers can be excluded from the measurement data to increase the quality of the sensitivity determination.

Simplified Linear Model of a Sensitivity Metric with Knowledge of a Target Refraction

In a further simplified, less individual model of the sensitivity metric, e.g. if only one measurement is available at a refractive deficit i=1, a relationship between the spherical and cylindrical refraction distance can be assumed according to equation (5):

$$\begin{matrix}{m}_{d} = m \\ {m}_{a} = f \cdot {m}_{d} = f \cdot m\end{matrix} \qquad ( 5 )$$

Here, the parameter f may be derived from empirical values and may be, e.g., a scalar. With the assumption according to equation (5), the system of equations (2) is simplified to form the following equation (6):

$$l g {v}_{i} = m \cdot ( {d}_{i} + f \cdot {a}_{i} ) + l g {v}_{0} {lgv}_{i} = m \cdot ( {a}_{i} + f \cdot {d}_{i} ) + {lgv}_{0} \qquad ( 6 )$$

Thus, the sensitivity m can be ascertained from a measurement with a refraction deficit i from equation (7):

$$m = \frac{{a}_{i} + f \cdot \vert {d}_{i} \vert}{{lgv}_{i} - {lgv}_{0}} \qquad ( 7 )$$

A value for f may be derived from relevant specialist literature, e.g. f=½ may be set, derived from Applegate, R. A, Sarver, E. J, Khemsara: “Are all aberrations equal?”, J Refract Surg. 2002, 18: pages 556-562. Or f=1 may be set, derived from Atchison et al.: “Blur limits for defocus, astigmatism and trefoil”, VisionResearch, 2009.

Here, a linear relationship does not necessarily have to be assumed for equation (5). Alternatively, more complex relationships may be established and the sensitivity derived therefrom, e.g. depending on a number of independent parameters and/or the refraction measurements—by substituting them into appropriately resolved relationships, cf. equations (4) and (7). The sensitivity may also be derived from a fitting method, such as least squares.

Other Models of a Sensitivity Metric with Knowledge of the Subjective Refraction

The sensitivity may also be calculated on the basis of another model. For example, from R. Blendowske, Unaided Visual Acuity and Blur: “A Simple Model”, Optometry and Vision Science, Vol. 92, No. 6, 2015, models are known which are characterized by particular simplicity and are based on only a few parameters. Such simple models are particularly suitable for calculating sensitivity and for fitting when data is scarce, for example because overfitting can be well avoided.

If a larger number of parameters are individually available, a model with many different parameters is more suitable, as described in DE 10 2017 007 663 A1, for example.

In principle, a plurality of different models may be used. Here, the model used in individual cases may depend on the number of visual acuity-refraction value pairs provided or ascertained. With a sufficient number of visual acuity-refraction value pairs, relatively complex, not necessarily linear models can be set up, the parameters of which can be adapted to the measurements.

The models listed above as examples may be generalized, e.g. in that a function describing the acuity of vision in the power vector space has contours of constant acuity of vision which correspond to ellipsoids or ovoids containing the point of maximum acuity of vision. This may be done analogously to a method set out in A. Rubin and W. F. Harris: “Closed Surfaces of Constant Visual Acuity in Symmetric Dioptric Power Space”, Optometry and Vision Science, Vol. 78, No. 10, 2001. Here, axial ratios may differ individually in a range from 0.25 to 4. Instead of individually measured values, mean values, medians or other estimated values of the corresponding model parameters of the population may also be used to calculate the acuity of vision.

In an exemplary embodiment, a generalization of the above equation (6) leads to different factors f, e.g. to equation (8):

$$l g {v}_{i} = {\{ ( {d}_{i} , {a}_{i}^{o r t} , {a}_{i}^{o b l} ) R [ \begin{matrix}{m}_{1}^{2} & 0 & 0 \\ 0 & {m}_{2}^{2} & 0 \\ 0 & 0 & {m}_{3}^{2}\end{matrix} ] {{R}^{T} ( {d}_{i} , {a}_{i}^{o r t} , {a}_{i}^{o b l} )}^{T} \}}^{1 / 2} + l g {v}_{0} \qquad ( 8 )$$

Here,

$${a}_{i}^{o r t} and {a}_{i}^{o b l}$$

denote the astigmatism of the refraction deficit with orthogonal (J0) and oblique (J45) cylinder axes and are defined as:

$$\begin{matrix}{a}_{i}^{o r t} = - \frac{{c}_{i}}{2} cos ( 2 {α}_{i} ) + \frac{{c}_{0}}{2} cos ( 2 {α}_{0} ) and \\ {a}_{i}^{o b l} = - \frac{{c}_{i}}{2} sin ( 2 {α}_{i} ) + \frac{{c}_{0}}{2} sin ( 2 {α}_{0} ) .\end{matrix}$$

Here, R represents a rotation matrix which determines the orientation of an ellipsoid of constant acuity of vision in the power vector space of the vectors

$$( {d}_{i} , {a}_{i}^{o r t} , {a}_{i}^{o b l} ) .$$

The eigenvalues m₁, m₂, m₃ denote the sensitivities to fogging in the direction of the first, second and third column vectors of the rotation matrix R in the power vector space.
Embodiments of Models of a Sensitivity Metric without Knowledge of the Target Refraction

In some embodiments, the ascertainment of the sensitivity may be performed without knowledge or ascertainment of the target refraction. This may be done if an associated refraction or visual acuity limit refraction is ascertained for several specified different visual acuity values. In this case, the best refraction or target refraction may be ascertained from the resulting measurement data. Furthermore, an actually ascertained best refraction may be checked from the measurement data by means of a model of a sensitivity metric.

Here, it can be assumed that a fogging, i.e. a deliberate refraction deficit, towards minus can be compensated by the test person by accommodation of the at least one eye. In this case, in the linear model according to the above equations (2) and (6) a point may be selected at which the visual acuity curve bends. In non-linear models where saturation occurs, the best refraction may be calculated directly as a parameter of the system of equations. For this purpose, the refraction deficit, i.e. the distances di and at; must be replaced by the difference between the best refraction and the set or applied correction in the corresponding formulas, i.e. in particular in equation (1).

The embodiments of models of a sensitivity metric explained above constitute examples to illustrate how sensitivity can be ascertained within the scope of the present invention.

The target may in particular be a real target (or real object) or a virtual target (or virtual object). In particular, the target may be a real object or a virtually projected object (or a projected virtual object). A target may, for example, be realized by a display (e.g. with one or more lenses and/or with one or more mirrors), by a light field display, and/or by a Badal optometer (which allows for a constant magnification despite a change in the power) and may be projected into the at least one eye of the test person.

A “virtual object” or “virtual target” is in particular understood to be an optical imaging system which generates wavefronts emanating from virtual object points so that these are incident on the at least one eye of the test person. Here, the wavefronts generated by the virtual target (each corresponding to a virtual object point) and incident on the at least one eye of the test person may have an adjustable spherical curvature and/or an adjustable cylindrical curvature component, wherein the cylindrical curvature component is preferably adjustable both in terms of the amount of curvature and in terms of the cylinder axis.

Preferably, the virtual position of the virtual object (target) may be changed so that different accommodation states of the at least one eye can be stimulated in this way. In particular, the position of the virtual object may preferably be changed between a position for stimulating far accommodation and a position for stimulating near accommodation. In addition, the position of the virtual object may preferably be set such that the at least one eye of the test person is no longer able to accommodate to the virtual object. In this case, the virtual object (target) can only be perceived by the test person as blurred in all directions. As a result, the ciliary muscles relax. Such a state is referred to as a “fogged” state.

A target is projected with an adjustable or variable target refraction (or target power) into the at least one eye of the test person. This projection may be performed with the aid of an optical system with which the power or refraction of the target, i.e. the target refraction, can also be adjusted and/or varied. Within the scope of the present invention, the “target refraction” is thus understood to mean the refraction (in particular spherical and/or astigmatic refraction) (applied or caused by the optical system) with which the target is projected into the at least one eye of the test person or with which the target is placed in front of the at least one eye of the test person.

In particular, an optical projection into or onto the test person's eye is regarded as a target such that this projection generates an image on the retina of the eye which corresponds to the image of a real object at a certain distance from the eye. This specific distance is also referred to here as a virtual position for the virtual target. In other words, a target within the meaning of this description is in particular an image of an object into the at least one eye of the test person. For example, a backlit slide may be used as the object. Since, in the case of a virtual target, the target is not (directly) a real object at the virtual position, a virtual position beyond infinity may also be simulated by suitable construction of the optical system for projection. This then corresponds to wavefronts which converge towards the eye (i.e. in the propagation direction).

The projection of a target (in particular a virtual target) into the at least one eye of the test person with the aid of an optical system is known in principle, so that it is therefore not discussed in more detail in the context of the present invention. For example, the projection of a target into the at least one eye of the test person is described in K. Nicke and S. Trumm: (“Brillengläser der Zukunft—Schritt 3 Der DNEye Scanner”) “Spectacle lenses of the future—Step 3 The DNEye Scanner”, Der Augenoptiker, June 2012, or also in the publication DE 10 2013 000 295 A1.

The target projected into the at least one eye of the test person is designed to verify a specified, in particular predetermined and/or known, visual acuity (or a specified visual acuity level). Here, “verify a specified visual acuity” is to be understood to mean in particular that with the aid of the target it may be ascertained or detected (in particular on the basis of a test person action) whether the at least one eye of the test person attains the specified visual acuity or the specified visual acuity level. In other words, the target specifies a certain visual acuity or a certain visual acuity level, the attainability of which may be detected for the at least one eye of the test person (in particular on the basis of a test person action). In particular, the target is designed (in particular dimensioned) in such a way that the virtual target can be or is assigned a specified visual acuity or a specified visual acuity level. In other words, the target is a target with a specified visual acuity or a specified visual acuity level. This means that the test person recognizes or can identify the target, in particular with an ideal refraction or with correction of any visual defect of the at least one eye of the test person, if the at least one eye of the test person attains or has at least the visual acuity or visual acuity level specified by the target. The visual acuity level is associated to and/or dependent on the dimension of the directional feature of the adapted visual sign.

In particular, the target may comprise or be an adapted visual sign suitable for determining the visual acuity. Here, the dimension or size of the directional feature of the visual sign depends on the specified visual acuity or the specified visual acuity level. In particular, the dimension or size of the directional feature of the visual sign is selected such that only a test person with a visual acuity which corresponds at least to the specified visual acuity or the specified visual acuity level can recognize and/or identify the directional feature of the visual sign.

The target may also be an image or photograph which contains two or more details, the recognition of which may be assigned to a specified visual acuity or a specified visual acuity level. The image may in particular represent objects (such as a road leading to infinity, a sky, a distant balloon, etc.), which can evoke a feeling of distance in the viewer. The above-mentioned details contained in the image (such as symbols or strips of fabric on a hot air balloon or the basket of a hot air balloon, clouds or symbols on clouds, lines on a road, symbols on roadside signs, etc.) are expressly included in the term visual sign in the context of this description. A particularly suitable symbol as a visual sign comprises, e.g., one or more concentric rings which merge into a circle at a given blur.

As is well known, the ascertainment of the visual acuity or visual acuity levels of a target or visual sign may be performed, e.g., by calculating the visual angle of details or by recognizing test persons with known acuity-of-vision characteristics.

After projecting the target into the at least one eye of the test person, a visual acuity limit refraction of the at least one eye of the test person associated with the specified visual acuity or the specified visual acuity level is ascertained.

The “visual acuity limit refraction” or “visual acuity level limit refraction” is understood to be the refraction or limit refraction at or above which the identifiability of the target for the test person changes. In particular, the “visual acuity limit refraction” or “visual acuity level limit refraction” is understood to be the refraction or limit refraction at which the target held up to the test person or the virtual target projected into his/her at least one eye, which is characterized by a specified visual acuity or a specified visual acuity level,
a) can be recognized and/or identified by him/her for the first time from a fogged state by varying the target refraction (applied or caused by the optical system), or
b) can just barely no longer be recognized and/or identified by him/her from an unfogged state by varying the target refraction (applied or caused by the optical system).

The visual acuity limit refraction is ascertained by varying the target refraction of the target projected into the at least one eye of the test person and by acquiring a test person action (e.g. a message or an input from the test person, in particular operating a button or a joystick). The variation of the target refraction may be performed gradually or preferably continuously. Preferably, the variation of the target refraction is monotonic and/or continuous. With the test person action, it is signaled or detected that the identifiability of the target has changed for the test person at the time of the test person action. In other words, the test person signals, via the test person action, that he/she can recognize or identify the target for the first time at the target refraction present or applied at the time of the test person action, or that he/she can just barely no longer recognize or identify the target for the first time. In particular, the visual acuity limit refraction corresponds to the target refraction or target power present or applied by the optical system at the time of the test person action.

Thus, the sensitivity of the at least one eye of the test person is determined, taking into account the specified visual acuity or the specified visual acuity level and the ascertained associated visual acuity limit refraction. For this purpose, adapted visual signs may be used whose dimension of the directional feature is assigned to the specified visual acuity values or the specified visual acuity levels.

The method may be performed in particular as part of autorefractometric or aberrometric measurements. For this purpose, at least one pair of a visual acuity level and the associated applied power is acquired. This is done by a signal from the test person during the change in the applied power for a target with a defined visual acuity level (i.e. defined size of a visual sign).

As already mentioned, at least two pairs of a visual acuity level and the associated applied power are required to ascertain the sensitivity. In conventional methods, the visual acuity level respectively attained by the test person with these powers (i.e. from which size the test person still recognizes visual signs) is ascertained for defined applied powers. In this variant of the method, however, the dimension of the directional feature of the adapted visual sign (and thus the visual acuity level) remains constant for at least one of these pairs and the applied power is changed. The test person signals when he/she can just barely still or no longer recognize an adapted visual sign of a defined size.

In contrast to the prior art, this alternative does not require the visual acuity level for a specific applied power—with a priori known or a priori unknown refraction deficit—to ascertain the sensitivity, but rather the applied power required to attain a specified visual acuity.

This procedure allows to ascertain the sensitivity quickly and easily. In particular, the procedure allows to ascertain the sensitivity (as a subjective measured variable) easily and without great additional effort during a normal objective refraction measurement. In particular, elaborate measurements during a subjective refraction can be avoided and the psychologically unfavorable step in which the test person is provided with a poorer correction after determining the best refraction and is required to solve visual tasks therewith can be dispensed with. In addition, the procedure may advantageously be combined very well with further measurements for determining individual parameters for advanced spectacle lenses (e.g. near measurement, pupillometry, keratography) and for optometric or ophthalmologic screening or with measurements to produce findings (such as keratography, opacity, pachymetry, tomography, tonometry or retinal images).

In one embodiment, an objective and/or subjective refraction result (in particular a combined refraction result based on an objective and subjective measurement including other data such as lower and/or higher order aberrations from aberrometry or other biometric data such as the shape of the cornea, distance between the lens and the retina, anterior chamber depth, etc.) of the at least one eye of the test person is ascertained before the step of projecting a target, which is designed to verify (or detect) a specified visual acuity, into the at least one eye of the test person. A “refraction result” is understood to be in particular an ascertained refraction value. In this way, in contrast to the previous procedure, the ascertainment of the sensitivity may be combined with one or more aberrometric or autorefractometric measurements. In particular, the ascertainment of the visual acuity may be combined with the measurement of autorefractometric or aberrometric data in the non-accommodated and accommodated state.

Preferably, the objective refraction value or the objective refraction result is ascertained in a fogged state. For this purpose, the test person may be presented with a target (e.g. an image or photo) or a corresponding virtual target may be projected into the at least one eye of the test person (with the aid of the optical system), which has a power that results in the test person only being able to recognize the target as blurred (or not completely sharply), whereby a relaxation of the ciliary muscle of the at least one eye of the test person is achieved. Such fogging may be carried out, e.g., with an additional power of approximately 1.25 dpt to 1.5 dpt as compared to the optimal refraction of the at least one eye of the test person.

In a special embodiment, the accommodation state of the eye may additionally be tracked in order to obtain even more reliable values for the sensitivity.

Preferably, before the step of varying the target refraction, the target is projected into the at least one eye of the test person with such a starting target refraction, i.e. the optical power first applied at least in the selected preferential direction, in such a way that the test person can only recognize and/or identify the target as blurred (or not completely sharply). In other words, a starting target refraction is preferably selected so that the test person cannot focus on the target or adapted visual signs by accommodation. This is achieved in particular by shifting the starting target refraction in the plus direction as compared to the optimal refraction of the at least one eye of the test person. Only by changing the target refraction in the minus direction can a state be achieved in which the test person can recognize and/or identify the target or visual signs. This has the additional advantage that the test person does not know the target or visual sign at first and is thus more likely to perform the test person action at the right time, namely only when he/she can actually identify the target or visual sign. If, on the other hand, the test person already knows the target or visual sign in advance or at the beginning of the measurement (due to a corresponding starting target refraction with which he/she sees the target or visual sign sharply), it was recognized in the context of the present invention that such a procedure is possible as an alternative, but may be inferior to the above-mentioned preferred embodiment in terms of the accuracy and reliability of the method. This is because a test person who already knows the target or visual sign in advance often tends to signal somewhat too late the point in time at or from which he/she just barely no longer recognizes and/or can no longer identify the target or visual sign after varying the target refraction in the plus direction.

In a further embodiment, the method comprises, either before or after the steps of projecting a target which is designed to verify a specified visual acuity, into the at least one eye of the test person and ascertaining a visual acuity limit refraction associated with the specified visual acuity of the target, ascertaining an optimal refraction (target refraction) of the at least one eye of the test person. In particular, the method may comprise ascertaining an objective and/or subjective refraction or an objective and/or subjective refraction result. Ascertaining an optimal refraction may also comprise ascertaining a combined refraction or a combined refraction result on the basis of an objective and/or subjective refraction measurement in which in particular further data such as lower and/or higher order aberrations from aberrometry or further biometric data such as the shape of the cornea, distance between the lens and the retina, anterior chamber depth, etc.) of the at least one eye of the test person are also taken into account. In this sense, the terms “refraction” and “target refraction” (or “refraction result”) in the context of “optimal refraction” are not to be limited to corrections of low order aberrations (e.g. sphere and astigmatism) but may also comprise higher order aberrations. Therefore, the term “refraction” could also be understood to mean “correction” in general. Preferably, the optimal refraction of the at least one eye of the test person is ascertained in a fogged state which may be achieved by holding up a corresponding target or projecting a corresponding target into the at least one eye of the test person (see above). Furthermore, according to this preferred embodiment, the visual acuity is determined which is attained by the at least one eye of the test person when compensating for any visual defect of the at least one eye of the test person (e.g. on the basis of an ascertained optimal refraction). In other words, the visual acuity is determined after the visual defect ascertained by the refraction measurement has been substantially corrected with the aid of an optical system or with the aid of lenses whose power corresponds to the ascertained refraction result, i.e. the visual acuity cum correctione (VCC). The determination of the visual acuity may be carried out using known methods. In particular, the ascertained optimal refraction and the measured associated visual acuity represent one of the at least two pairs of visual acuity-refraction values provided, which are used or taken into account when ascertaining the sensitivity. In this way, it is possible to combine the ascertainment of the sensitivity with measurements of the objective and/or subjective refraction or to integrate it into such measurements. The sensitivity can thus be ascertained quickly and easily, in particular in combination with other measurements.

In a further embodiment, the method, preferably after projecting a target which is designed to verify a specified visual acuity, into the at least one eye of the test person and after ascertaining a visual acuity limit refraction associated with the specified visual acuity of the target, further comprises the steps of:
ascertaining a subjective refraction result or a subjective refraction for the at least one eye of the test person;
determining the visual acuity attained by the at least one eye of the test person when compensating for any visual defect of the at least one eye of the test person on the basis of the ascertained subjective refraction result.

The ascertained subjective refraction and the visual acuity of the at least one eye of the test person determined at this ascertained subjective refraction preferably represent one (or a further, in particular a second, third, fourth, etc.) of the visual acuity-refraction value pairs for determining the sensitivity provided by the method.

Furthermore, the method preferably comprises ascertaining an optimal refraction of the at least one eye of the test person on the basis of the subjective refraction result and an objective refraction result. The optimal refraction is in particular a combined refraction from the subjective and objective refraction result. Ascertaining a combined refraction result from an objective and subjective refraction measurement is known in principle and is therefore not explained in more detail in the context of the present description. For example, a combined refraction may be ascertained by first carrying out an objective refraction measurement and then adapting the objective refraction result with the aid of a subjective refraction performed subsequently thereto. In particular, it is also possible to ascertain a combined refraction by averaging the objective and subjective refraction.

In a further embodiment, the sensitivity is determined on the basis of at least one calculated refraction deficit, wherein the at least one calculated refraction deficit is calculated on the basis of an ascertained optimal refraction. Here, the optimal refraction may be an ascertained objective and/or subjective refraction. In particular, the optimal refraction may be a combined refraction from an objective and subjective refraction.

Preferably, the refraction deficit is determined “ex-post”, i.e. only after projecting a target, which is designed to verify a specified visual acuity, into the at least one eye of the test person and after ascertaining a visual acuity limit refraction associated with the specified visual acuity of the target. Preferably, the refraction deficit is determined only after ascertaining at least one visual acuity-refraction value pair. Preferably, the refraction deficit is determined after performing an objective and/or subjective refraction measurement, and in particular after ascertaining an ideal refraction or an ideal refraction result from an objective and subjective refraction measurement. For example, in a preferred embodiment, the following steps may be carried out, in particular in the stated order:
1) Performing an objective refraction measurement (as part of the procedure according to the invention);
2) ascertaining at least one visual acuity-refraction value pair (as part of the procedure according to the invention);
3) performing a subjective refraction measurement;
4) ascertaining an ideal refraction or an ideal refraction result from the objective and subjective refraction measurement; and
5) calculating the refraction deficits and the sensitivity starting from the result from step 4, i.e. on the basis of the ascertained ideal refraction or the ascertained ideal refraction result.

In a further embodiment, varying the target refraction comprises monotonically decreasing the target refraction and/or monotonically increasing the target refraction.

In a further embodiment, ascertaining a visual acuity limit refraction of the at least one eye of the test person associated with the specified visual acuity is carried out by decreasing the target refraction and acquiring a test person action while decreasing the target refraction, and/or by increasing the target refraction and acquiring a test person action while increasing the target refraction, wherein it is detected with each test person action that the identifiability of the target for the test person has changed at the time of the respective test person action. In this way, the “blur point” is approached from different directions. In other words, one blur point may be determined when increasing and another blur point when decreasing the target refraction. These blur points may be different from each other and subsequently averaged. In particular, the sensitivity may be determined as part of a minimization of the error squares by means of known metrics from both blur points.

In a further embodiment, at least two of the provided visual acuity-refraction value pairs are provided by the following steps:
projecting a first target with a first adjustable and/or variable target refraction into the at least one eye of the test person, wherein the first target is designed to verify a specified (predetermined and/or known) first visual acuity (or a specified first visual acuity level);
ascertaining a first visual acuity limit refraction of the at least one eye of the test person associated with the specified first visual acuity (or the specified first visual acuity level) by varying (in particular continuously, monotonically and/or constantly varying) the first target refraction of the first target projected into the at least one eye of the test person and acquiring a first test person action by which it is signaled or detected that the identifiability of the first target for the test person has changed at the time of the first test person action;
projecting a second target with a second adjustable and/or variable target refraction into the at least one eye of the test person, wherein the second target is designed to verify a specified (predetermined and/or known) second visual acuity (or a specified second visual acuity level) different from the specified first visual acuity (or the specified first visual acuity level);
ascertaining a second visual acuity limit refraction of the at least one eye of the test person associated with the specified second visual acuity (or the specified second visual acuity level) by varying (in particular continuously, monotonically and/or constantly varying) the second target refraction of the second target projected into the at least one eye of the test person and acquiring a second test person action by which it is signaled or detected that the identifiability of the second target for the test person has changed at the time of the second test person action.

In particular, determining the sensitivity of the at least one eye of the test person is carried out using or taking into account the specified first visual acuity and the ascertained associated first visual acuity limit refraction, and further using or taking into account the specified second visual acuity and the ascertained associated second visual acuity limit refraction. Preferably, the first specified visual acuity or the first specified visual acuity level of the first target is smaller than the second specified visual acuity or the second specified visual acuity level of the second target. For example, the first specified visual acuity or the first specified visual acuity level may have the value of 0.8 log Mar, while the second specified visual acuity or the second specified visual acuity level has the value of 1.0 log Mar. Or, for example, the first specified visual acuity or the first specified visual acuity level may have the value of 0.4 log Mar, while the second specified visual acuity or the second specified visual acuity level has the value of 0.8 log Mar or 1.0 log Mar. It is to be understood that other values may also be selected. Preferably, the change in the specified visual acuity or the specified visual acuity level from one virtual target to the next target is in the range from 0.2 log Mar to 0.7 log Mar, preferably in the range from 0.2 log Mar to 0.5 log Mar, and particularly preferably in the range from 0.2 log Mar to 0.3 log Mar.

In a further embodiment, ascertaining a visual acuity limit refraction comprises measuring and/or monitoring an accommodation state of the at least one eye of the test person, wherein measuring the accommodation state is carried out in particular at least at the time of or immediately after the test person action. The results of such a measurement or monitoring may be used to control the procedure (e.g. aborting or repeating individual steps in the event of unwanted accommodation (e.g. exceeding a certain threshold)). The measurement may be performed either continuously or only during or immediately after the test person action. Furthermore, an accommodation state (sphere, cylinder, lower or higher order aberration) measured, ideally at the time of the test person action, may be included in the calculation of the sensitivity or the refraction deficit. For example, the amount of accommodation may be subtracted from the amount of the distance of the applied power from the refraction value for distance. Expressed in formulas, the following applies in the simplest case: The sensitivity represents the visual acuity V as a function f of the refraction deficit F, i.e. V=f(F). Here, the refraction deficit F
without accommodation is the difference between the actually applied power T and the ideal power I, i.e. F=T−I; and
with accommodation is the difference between the actually applied power T and the currently measured power Gₐ, i.e. F=T−Gₐ.

If there is a deviation D between the ideal power I and the measured power with the eye relaxed (power G₀), the following applies: 1=G₀+D. Accordingly, in this situation F=T−(G₀+D) or F=T−(Gₐ+D). For values of the sphere, this formula can be used as described. For cylindrical values, the cross-cylinder formula is to be used accordingly. Zernike coefficients (also for higher order aberrations) or power vectors may be used analogously.

Alternatively or additionally, ascertaining a visual acuity limit refraction may comprise measuring and/or monitoring a pupil size (e.g. pupil radius) of the at least one eye of the test person, wherein measuring the pupil size is carried out in particular at least at the time of or immediately after the test person action. The pupil size may be measured, e.g., by means of a camera which is part of a refraction unit, e.g. an autorefractometer or aberrometer, or by means of a separate camera. The pupil size measured at the time of the test person action (i.e. at the blur point) or correspondingly shortly before or after (e.g. up to 2 seconds before reaching the blur point) may be used in determining the sensitivity of the at least one eye of the test person to blur. In particular, the measured pupil size may be used to quantify the blur of the image on the retina, preferably with the aid of a suitably parameterized eye model and a known additional fogging. Instead of a complete eye model, a simpler description may also be used. For example, the angle may be calculated at which the dispersion disk of a point presented as blurred dot can be observed for a given pupil and given additional fogging (see, e.g., WO 2019 034525 A1). The sensitivity may be determined in the context of such a visual acuity model as a deterioration in visual acuity per angle of the dispersion disk.

In a further embodiment, in order to ascertain a visual acuity limit refraction, the test person is presented with a visual task with at least two, preferably at least three, particularly preferably at least four, in particular four or eight, possible different answers, wherein the test person can answer the visual task using the test person action. A “visual task” is understood here in particular to be a task which has a specified and therefore verifiable solution. In particular, the visual task is therefore a verifiable task (i.e. a visual task whose solution is known and therefore verifiable). In other words, the test person action goes beyond a mere communication about the recognizability or identifiability of the target. Preferably, the visual task is based on a forced choice, i.e. the test person is “forced” to make a selection from several or at least two or a plurality of possible answers, wherein the correct answer is preferably specified or known. Here, such a visual task is referred to as a “forced choice” visual task. Solving the visual task or making a selection may be done using a joystick, e.g., with which the test person can operate different directions. For example, the visual task may consist of the test person having to identify the position or direction of the gap in an adapted visual sign using a joystick. If the adapted visual sign is, e.g., a Landolt ring, there are two possible positions for this and thus two possible answers for the test person as to how the directional feature can be arranged in parallel to the selected preferential direction. It is to be understood that, in principle, other visual signs may also be used so that the test person has answers which depend on the adapted visual sign. In this way, the method is more accurate and reliable than if the test person only has to give an unverified response (e.g. “yes” or “no” or “recognizable” or “not recognizable”).

In a further embodiment, prior to the step of ascertaining a visual acuity limit refraction, first aberrometric data of the at least one eye of the test person is acquired, preferably for a far accommodation state and/or a fogged state of the at least one eye of the test person and in particular at a first brightness. Furthermore, the method preferably comprises acquiring second aberrometric data of the at least one eye of the test person for a near accommodation state of the at least one eye of the test person, in particular at a second brightness whose value is below that of the first brightness. Here, acquiring second aberrometric data is preferably carried out prior to the step of ascertaining a visual acuity limit refraction. In the context of this description, “aberrometric data” (or “aberrometric measurements”) refers to data for describing the aberrations of an eye (measurements for obtaining this data), the information content of which corresponds at least to the term of the order “defocus” when represented with Zernike coefficients but ideally includes higher orders (e.g. coma and spherical aberrations). In particular, the “aberrometric data” may also comprise or be (purely) autorefractometric data. In particular, acquiring aberrometric data also comprises acquiring (purely) autorefractometric data (i.e. sphere and/or cylinder and/or axis). The first and second brightness is preferably each provided as a brightness in the regime of mesopic vision (preferred luminance in the range from about 0.003 cd/m² to about 30 cd/m², particularly preferably in the range from about 0.003 cd/m² to about 3 cd/m², even more preferably in the range from about 0.003 cd/m² to about 0.3 cd/m², most preferably in the range from about 0.003 cd/m² to about 0.03 cd/m²). Here, in particular, brightness is always understood as the brightness at the location of the eye or the brightness to be acquired by the eye.

Together with acquiring first aberrometric data and/or acquiring second aberrometric data (i.e. in particular at the first or second brightness and at the first or second accommodation state), first or second pupillometric data may further be acquired for the at least one eye of the test person. Here, the term “pupillometric data” (or pupillometric measurements) refers to information on the size of the pupil (or measurements to obtain this data) which comprise at least one size specification (for example in the form of a radius) but may also reflect the shape of the pupil in a more complex form. In addition, the pupillometric data may contain information on the position of the pupil (for example relative to the apex of the corneal vertex or the optical axis of the eye).

A further aspect relates to a method for calculating, optimizing or evaluating a spectacle lens for at least one eye of a test person or spectacle wearer, taking into account the sensitivity of the at least one eye of the test person, wherein the sensitivity of the at least one eye of the test person is ascertained by one of the methods according to the invention.

In particular, the method for calculating, optimizing or evaluating a spectacle lens for at least one eye of a test person may comprise the following steps:
a) Providing an assignment of at least one imaging property or aberration of a spectacle lens system to the visual acuity of the spectacle wearer or an average spectacle wearer when viewing an object through the spectacle lens system;
b) determining or specifying a target function for the spectacle lens to be calculated or evaluated, in which the assignment from step (a) is to be evaluated;
c) calculating or evaluating the spectacle lens to be calculated or evaluated by evaluating the target function, wherein the target function is evaluated at least once.

The assignment of the at least one imaging property or aberration of a spectacle lens system to the visual acuity of the spectacle wearer may depend parametrically on the measured initial visual acuity and/or the measured sensitivity of the spectacle wearer.

Calculating and/or optimizing the spectacle lens may in particular comprise minimizing or maximizing the target function. The method for calculating, optimizing or evaluating a spectacle lens may further comprise tracing at least one light beam emanating from the object for at least one gaze direction with the aid of wavefront tracing, ray tracing or wavefield tracing through the spectacle lens system and/or through the spectacle lens to be calculated or evaluated up to an evaluation surface in the spectacle lens system. Furthermore, the method for calculating, optimizing or evaluating a spectacle lens may comprise calculating the difference, present at the evaluation surface, in the light beam emanating from the object as compared to a reference light beam converging on the retina of a model eye and determining the at least one imaging property or aberration based on the calculated difference. Tracing at least one light beam emanating from the object is preferably carried out by means of wavefront tracing, wherein calculating the difference present at the evaluation surface comprises calculating the wavefront difference between the wavefront of the light beam emanating from the object and the wavefront of the reference light beam converging on the retina, wherein the wavefront difference at the evaluation surface is calculated. Furthermore, the method for calculating, optimizing or evaluating a spectacle lens may comprise assigning a geometrical-optical angle and/or a square shape in the space of geometrical-optical angles to the calculated wavefront difference, wherein the at least one imaging property or aberration depends on at least one component of the geometrical-optical angle and/or the square shape.

Alternatively or additionally, the method for calculating, optimizing or evaluating a spectacle lens may comprise the following steps:
Specifying a first surface and a second surface for the spectacle lens to be calculated or optimized;
Ascertaining the course of a main ray through at least one viewing point of at least one surface of the spectacle lens to be calculated or optimized into a model eye;
evaluating an aberration of a wavefront, resulting along the main ray from a spherical wavefront incident on the first surface of the spectacle lens, at an evaluation surface as compared to a wavefront converging at a point on the retina of the eye model;
iteratively varying the at least one surface of the spectacle lens to be calculated or optimized until the evaluated aberration corresponds to a specified target aberration.

A further aspect relates to a method for manufacturing a spectacle lens, comprising:
calculating or optimizing a spectacle lens according to the method according to the invention for calculating or optimizing a spectacle lens; and
manufacturing the thus calculated or optimized spectacle lens.

Furthermore, the invention provides a computer program product, in particular in the form of a storage medium or a data stream, containing a program code which, when loaded and executed on a computer, is designed to perform a method according to the invention, in particular for ascertaining the sensitivity of at least one eye of a test person and/or for calculating, optimizing or evaluating a spectacle lens and/or for manufacturing a spectacle lens. In other words, the invention provides a computer program product which comprises machine-readable program code which, when loaded on a computer, is suitable for executing the method according to the invention described above. In particular, a computer program product is to be understood as a program stored on a data carrier. In particular, the program code is stored on a data carrier. In other words, the computer program product comprises computer-readable instructions which, when loaded into a memory of a computer and executed by the computer, cause the computer to perform a method according to the invention.

In particular, the invention provides a computer program product containing a program code which, when loaded and executed on a computer, is designed and configured to perform a method according to the invention for ascertaining the sensitivity of at least one eye of a test person and/or a method according to the invention for calculating, optimizing or evaluating a spectacle lens and/or a method according to the invention for manufacturing a spectacle lens.

A further aspect relates to an apparatus for determining the sensitivity of at least one eye of a test person, comprising:
a target provision device for providing a target which is designed to verify a specified visual acuity and configured to display at least one adapted visual sign;
an optical system for projecting the target with a target refraction into the at least one eye of the test person, wherein the optical system is designed to adjust and vary the target refraction;
a feedback unit for acquiring a test person action to detect that at the time of the test person action, in particular as a result of varying the target refraction of the target projected into the at least one eye of the test person with the aid of the optical system, the identifiability of the target for the test person has changed; and
a visual acuity limit refraction ascertaining unit for ascertaining a visual acuity limit refraction of the at least one eye of the test person associated with the specified visual acuity, wherein the visual acuity limit refraction ascertaining unit is designed to acquire (in particular to determine and store) the target refraction caused by the optical system at the time of the test person action.

The target provision device may comprise, e.g., an electronic display or a digital screen. In particular, the display may be designed so that individual pixels of the display, different areas or different components of the display can be controlled individually, in particular to display composite optotypes. For example, partial segments of a ring may be displayed, with which Landolt-C optotypes with differently directed openings can be generated or shown. Alternatively or additionally, complete optotypes such as letters or numbers may also be formed as entire and in particular switchable LCD elements. In general, the display is configured to display adapted visual signs.

The target provision device may, e.g., comprise a folding or sliding or rotating mechanism, for example magnetic or motorized, with which different targets or images can be displayed and/or exchanged. The targets or images may also be partially transparent and only contain areas which are to be shown in addition to another image.

Transparent, backlit images may also be designed such that certain parts of the image can only be seen when one or more specific light sources (e.g. in otherwise shadowed areas or with special wavelengths) are switched on or off.

The optical system is arranged in particular between the at least one eye of the test person and the target provision device or the provided target. The optical system may be configured as a refraction unit. The optical system is designed to apply or cause different target powers as optical powers at least in the selected preferential direction and thus influence the recognizability of the target for the at least one eye of the test person. Here, the optical system may be designed to hold up different spherical powers as optical powers. This may be done, e.g., by arranging one or more spherical lenses, for example in the form of a Badal system. Alternatively or additionally, one or more adaptive lenses, possibly in combination with conventional lenses, may be used or arranged. In more complex cases, the optical system may be designed to apply or cause various cylindrical powers or higher order powers in addition to or instead of spherical powers.

The optical system may have at least one lens having a spherical power and/or at least one lens having a cylindrical power. For example, the optical system may comprise a magazine with a plurality of spherical lenses and/or cylindrical lenses which each have different spherical or cylindrical powers, and wherein the magazine is designed and arranged such that individual spherical lenses or individual cylindrical lenses and/or a combination of several spherical lenses or cylindrical lenses of the magazine are selectable and used for projecting the target. The optical system may also have, e.g., an Alvarez lens system. In other words, the test person is presented with a target (or a projected or virtual target) through which the test person sees the target or virtual target. The optical system may also comprise, e.g., two lenses rotatable relative to each other, each with at least one cylindrical component in the powers. In particular, the optical system may have two cylindrical lenses with interlocking, rotationally symmetrical surfaces, preferably flat surfaces, facing each other. The optical system may also have a positive and a negative cylindrical lens with opposing equal powers, which are rotatably supported relative to each other and are preferably slidable relative to each other.

Furthermore, it is possible that the visual angle of the target changes upon application of different powers by the optical system. This may either be prevented by setting up the optical system accordingly or determined by calculation and compensated for in the representation. For this purpose, the visual angle must be determined depending the applied power and a visual acuity value must be assigned on the basis of this actual visual angle, which can be achieved, e.g., by determining the magnification of the optical system and a correspondingly reduced representation of the target. Alternatively, the optical system may be calibrated with the aid of a camera by directly realizing the size of the target with a camera arranged in place of the at least one eye of the test person (and looking into the optical system).

In principle, the feedback from the test person or the test person action may be verbal. In this case, a user may memorize the state of the optical system during the feedback or test person action and/or pass the feedback directly to the feedback system. However, this variant is prone to errors and causes delays. For this reason, direct feedback from the test person to the feedback system is preferred. In the simplest case, the feedback system may comprise a button for this purpose. In another preferred embodiment, the feedback system may also comprise two buttons (“+” and “−”), three buttons (“+”, “−” and “OK”), four buttons (e.g. “+”, “−”, “OK” and “Cancel”), etc., and/or a joystick. Alternatively or additionally, the feedback system may comprise a microphone for acquiring verbal utterances of the test person.

In one embodiment, the apparatus comprises an evaluation unit for determining the sensitivity of the at least one eye of the test person on the basis of at least two provided visual acuity-refraction value pairs. Here, the visual acuity limit refraction ascertaining unit may be a component of the evaluation unit. In other words, the evaluation unit may comprise the visual acuity limit refraction ascertaining unit.

In a further embodiment, the apparatus comprises an autorefractometric or aberrometric measurement unit for determining one or more objective refractions of the at least one eye of the test person, wherein the autorefractometric or aberrometric measurement unit is preferably designed to measure and/or monitor an accommodation state of the at least one eye of the test person. Furthermore, the autorefractometric or aberrometric measurement unit, as a refraction unit, may have a camera for ascertaining a pupil size (in particular a pupil radius) of the at least one eye of the test person. Alternatively or additionally, the autorefractometric or aberrometric measurement unit may comprise a calibration camera for calibrating the optical system. The camera for ascertaining a pupil size and the calibration camera may also be realized in a single camera which combines both functions (ascertaining the pupil size and calibrating the optical system).

In a further preferred embodiment, the apparatus comprises a pupil size measurement unit (in particular a camera) for ascertaining a pupil size (in particular a pupil radius) of the at least one eye of the test person. Alternatively or additionally, the apparatus may comprise an illumination device for generating at least two brightnesses. Alternatively or additionally, the apparatus may comprise a pupillometer device which is designed to acquire first pupillometric data of the at least one eye at a first brightness and to acquire secondary pupillometric data of the at least one eye at a second brightness.

A further aspect relates to an apparatus for calculating, optimizing or evaluating a spectacle lens for at least one eye of a test person, taking into account the sensitivity of the at least one eye of the test person, comprising an apparatus according to the invention for ascertaining the sensitivity of the at least one eye of the spectacle wearer.

The apparatus for calculating, optimizing or evaluating a spectacle lens may comprise in particular the following components:
a surface model database for specifying a first surface and a second surface for the spectacle lens to be calculated or optimized;
a main ray ascertaining module for ascertaining the course of a main ray through at least one viewing point of at least one surface of the spectacle lens to be calculated or optimized into a model eye;
an evaluation module for evaluating an aberration of a wavefront, resulting along the main ray from a spherical wavefront incident on the first surface of the spectacle lens, at an evaluation surface as compared to a wavefront converging at a point on the retina of the eye model; and
an optimization module for iteratively varying the at least one surface of the spectacle lens to be calculated or optimized until the evaluated aberration corresponds to a specified target aberration.

A further aspect relates to an apparatus for manufacturing a spectacle lens, comprising:
calculation or optimization means which are designed to calculate or optimize the spectacle lens according to a method according to the invention for calculating or optimizing a spectacle lens; and
processing means which are designed to process the spectacle lens according to the result of the calculation or optimization.

A further aspect relates to a spectacle lens manufactured by a method according to the invention for manufacturing a spectacle lens and/or by means of an apparatus according to the invention for manufacturing a spectacle lens.

Furthermore, the invention provides a use of a spectacle lens manufactured according to the manufacturing method according to the present invention, in particular in a preferred embodiment, in a specified average or individual use position of the spectacle lens in front of the eyes of a specific spectacle wearer for correcting a visual defect of the spectacle wearer.

In particular, a computer-implemented method according to the invention may be provided in the form of ordering and/or industry software. In particular, in such a method, the data required for the calculation and/or optimization and/or manufacture of a spectacle lens may be acquired and/or transmitted.

An apparatus according to the invention and/or a system according to the invention, e.g. for ordering a spectacle lens, may in particular comprise a computer and/or data server which is designed to communicate via a network (e.g. the Internet). The computer is in particular designed to execute a computer-implemented method, e.g. an ordering software for ordering at least one spectacle lens, and/or a transmission software for transmitting relevant data, and/or an ascertaining software for ascertaining relevant data, and/or a calculation or optimization software for calculating and/or optimizing a spectacle lens to be manufactured, according to the present invention.

It is to be understood that the features mentioned above and to be explained below are usable not only in the combination indicated in each case, but also on their own or in other combinations, without departing from the scope of the present invention.

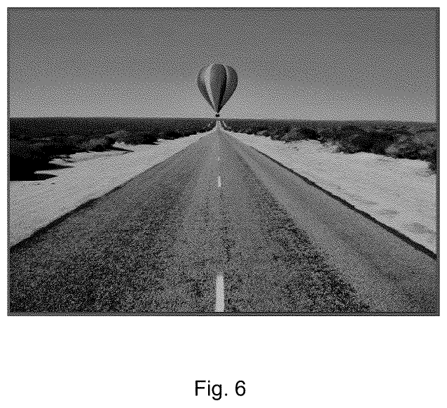

In the following, individual embodiments for achieving the object are described by way of example with reference to FIGS. 6-8. Here, the individual embodiments described partly have features which are not absolutely necessary to carry out the claimed subject matter but provide desired properties in certain applications. Thus, embodiments which do not have all the features of the embodiments described below are also to be regarded as falling within the scope of the technical teaching described. Furthermore, in order to avoid unnecessary repetition, certain features are mentioned only in relation to individual embodiments described below. It should be noted that the individual embodiments should therefore be considered not only in isolation, but also in combination. Based on this combination, a person skilled in the art will recognize that individual embodiments can also be modified by incorporating individual or several features of other embodiments. It should be noted that a systematic combination of the individual embodiments with individual or several features described in relation to other embodiments may be desirable and useful and should therefore be considered and also be regarded as included in the description.

FIG. 6 shows an exemplary image or photograph which includes a hot air balloon and a road and conveys a sense of distance to the viewer. Such an image may, e.g., be projected within the scope of the present invention as a target (in particular as a virtual target) into the at least one eye of a test person to perform an objective refraction measurement, for example in a fogged state in which the test person recognizes the image or details of the image only as blurred.

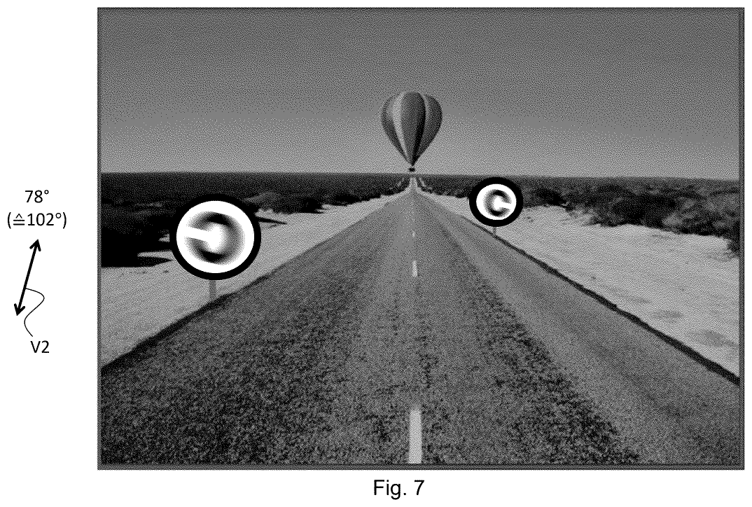

FIG. 7 shows the image or photo of FIG. 6 with exemplary adapted visual signs integrated in the image or superimposed on the image for a selected preferential direction. Each of these adapted visual signs has a specified visual acuity or a specified visual acuity level. As part of the method according to the invention, the image with the adapted visual signs is provided with an adjustable target refraction with the aid of an optical system. This target refraction is varied by means of the optical system and the test person signals by means of a test person action that the identifiability of the target or the adapted visual signs has changed for him/her at the time of the test person action. In this way, visual acuity-refraction value pairs can be provided to ascertain the sensitivity of the at least one eye of the test person.

One or more targets may be presented to the test person or projected as virtual targets into the at least one eye of the test person. Depending on the embodiment, two or more targets may be used, which may also be identical in content.

For example, a first target may be an image which conveys a sense of distance (see e.g. FIG. 6), a second target may be one or more adapted visual signs of a certain size, and a third target may be one or more adapted visual signs of a different size.

Alternatively, the first target may be an image which conveys a sense of distance, while the second and third targets may be identical in content and may contain one or more adapted visual signs, each in one of two sizes.

Alternatively, all three targets may be identical and represent an image which conveys a sense of distance, but contain one or more details, the recognition of each of which may be assigned to a visual acuity level. These details are explicitly included in the term adapted visual sign in this description. Examples of such details are in an image containing, e.g., a hot air balloon and a road:
symbols or fabric panels on the hot air balloon as well as the basket of a hot air balloon,
clouds or symbols on clouds,
lines on a road, and/or
symbols on roadside signs.

A particularly suitable symbol has, e.g., one or more concentric rings which merge into a circle at a given blur.

In these embodiments, in contrast to the prior art, the visual acuity level for a specific applied power is not ascertained to ascertain the sensitivity, but rather the applied power required to attain a specified visual acuity. Furthermore, the ascertainment of the visual acuity may be combined with the measurement of autorefractometric or aberrometric data in the non-accommodated and accommodated state. In a special embodiment, the accommodation state of the eye may also be tracked so as to obtain even more reliable values for the sensitivity.

A. Procedure According to an Exemplary Embodiment without Subjective Refraction

An examination on the test person may be performed, e.g., as follows:
1) With the aid of an autorefractometric or aberrometric measurement, the objective refraction value of the test person is determined. For this purpose, the test person is presented with a first target. Here, by a suitable optical system, the test person is presented with a first power which does not allow him/her to see the target completely sharply to achieve relaxation of the ciliary muscle in this way.
2) The test person is now presented with a second target and, with the aid of the optical system, a second power is applied at which a test person with high visual acuity cannot recognize the at least one adapted visual sign. This is achieved in particular by selecting a spherical power as the applied optical power, which corresponds to the mean sphere or one of the two principal meridians of the objective refraction value plus an additional positive optical power. The latter power—often called “fogging”—is selected because the test person cannot compensate for such a power by accommodation. Standard values on the basis of mean values for a plurality of test persons may be taken to set the rate at which the optical power held up is changed. For example, it is known that the visual acuity is approximately halved by 0.5 dpt spherical or 1 dpt cylinder with fogging. Preferably, the target refraction is varied as an applied optical power at a rate of between 1/16 dpt per second and ½ dpt per second. The additional optical power may also depend on the pupil measured by the aberrometry unit. It may be, e.g., reciprocally proportional to the pupil radius, so that test persons with smaller pupils are preferably fogged with a stronger power than test persons with larger pupils to ensure that the blur perceived by all test persons is similar.
3) Alternatively, a sphero-cylindrical power may be used as an optical power. For example, a cylindrical power for the optical system may be taken from the objective refraction and an additional positive spherical power may be applied to a mean objective refraction value. Alternatively or additionally, an astigmatic offset may be applied to the objective refraction value (so-called astigmatic fogging). The optical power is now changed slowly (e.g. between 1/16 dpt per second and ½ dpt per second) in the direction of optimal or objective refraction (by varying the spherical and/or astigmatic power).
4) As soon as the test person can recognize the directional feature of the adapted visual sign of the second target by changing the applied optical power, he/she communicates this (e.g. via the “OK” button). If necessary, he/she may set the limit power himself/herself (e.g. using the “+” and “−” buttons) and confirm it (e.g. also via the “OK” button). The power set thereby is saved as the “visual acuity limit power” or “visual acuity limit refraction” for this recognition of the second target.
5) The test person is presented with the third target.
6) The optical power is now further changed slowly (e.g. between 1/16 dpt per second and ½ dpt per second) in the direction of the optimal or objective refraction value (by varying the applied optical power).
7) As soon as the test person can recognize the directional feature of the adapted visual sign of the third target by changing the applied optical power, he/she communicates this (e.g. via the “OK” button). If necessary, he/she may set the limit power (e.g. using the “+” and “−” buttons) and confirm it (e.g. also via the “OK” button). The power set thereby is saved as the “visual acuity limit power” or “visual acuity limit refraction” for this recognition of the third target.

The sensitivity may be determined from the visual acuity levels of the two targets, more precisely the two respective dimensions of the directional features of the two adapted visual signs, the objective refraction value, the optical power applied during this recognition of the second target and the optical power applied during this recognition of the third target. For this purpose, in particular a sensitivity metric as described above in exemplary embodiments may be used. Here, the refraction deficits result from the (e.g. spherical and/or astigmatic) distance of the optical power applied for this recognition of the respective target from the objective refraction value.

B. Procedure According to an Exemplary Embodiment with Subjective Refraction

In this variant, steps 5) to 7) above from the procedure in section A may be omitted. Thus, only the visual acuity for a target and the optical power applied when recognizing (e.g. the directional features of) a target need to be determined. Thereafter, subjective refraction ascertainment is performed and the subjective refraction value and the visual acuity (visual acuity cum correctione, VCC) attained by the test person are determined therein. The objective refraction value may be used as a starting value for the subjective refraction ascertainment.

Alternatively, the subjective refraction ascertainment with visual acuity determination may be carried out prior to the steps in section A. In this case, the autorefraction or aberrometry and the determination of the objective refraction value (step 1) may be dispensed with and the subjective refraction value may be used instead.

Here, the refraction deficit may be calculated as the spherical or astigmatic distance of the power when recognizing the target from the subjective refraction value.

Instead of the subjective refraction value, a combined refraction value may also be used to calculate the sensitivity or refraction deficit. This may be calculated on the basis of the subjective refraction value and the objective refraction value or other data (e.g. lower or higher order aberrations from the aberrometry or other biometric data such as the shape of the cornea, distance between the lens and the retina, anterior chamber depth).

### C. Adapting the Visual Acuity Level of a Target

Furthermore, the at least one visual acuity level of the adapted visual sign or the adapted visual signs of a target may be adapted to the test person. This is useful, for example, if an astigmatism of the test person cannot be compensated for. The visual acuity level(s) of the (virtual) target may then be selected such that the target can still be recognized despite the residual refraction deficit due to the astigmatism.

Information about the acuity of vision (e.g. visual acuity cum correctionem or visual acuity sine correctionem, for example from the subjective refraction determination) may also be included in the ascertainment of the target size, i.e. the dimension of the directional feature of the adapted visual sign of the target.

If the test person does not recognize the directional feature of the adapted visual sign despite a small deviation of the applied optical power from the objective, subjective or combined refraction value, it is possible to switch to a lower visual acuity level and to repeat the corresponding step with a lower visual acuity level.

In addition or instead, the findings from step 4 may be included in the determination of the visual acuity level in step 6.

In order to avoid that the test person already knows the adapted visual sign during multiple measurements or when switching between the eyes, the at least one adapted visual sign or symbol or detail in the image may be changed between different measurements or when changing eyes. Here, in particular the selected preferential direction may be adapted to the astigmatism of the other eye, or a change may be made from an adapted Landolt ring to an adapted Snellen E. By their very nature, electronic displays are particularly well suited for this purpose as a target provision device.

### D. Finding the Blur Point and Setting the Power by the Test Person

### Finding the Blur Point

As an alternative to the procedure in the previous sections, the optical power held up at the beginning (i.e. in step 2) after section A or B) may also be a power which allows the recognition of the directional feature of the adapted visual sign of the target. This may be an objective, subjective or combined refraction value.

In steps 5) and 6), the optical power held up is then removed from this optical power in the plus direction. This direction is selected to prevent accommodation. In steps 4) and 7), the test person then signals the time at which he/she can no longer recognize the directional feature of the visual sign.

If the applied optical powers are determined for two visual acuity levels analogously to the procedure in section A, in this case the applied optical powers for the higher visual acuity level may be determined first (steps 2-4) and then (steps 5-7) for the lower visual acuity level. In this way, the refraction deficit can be increased in the course of the procedure, whereby first the directional feature of the adapted visual sign with the more difficult recognizability (higher visual acuity level) and then the one with easier recognizability (lower visual acuity level) becomes unrecognizable.

### Correcting the Focus and Blur Point

Optionally, in steps 4) and 7) of the above embodiments, the test person may correct the optical power held up (i.e. applied) if he/she is not sure that he/she has signaled the correct time or the correct optical power held up. This may be done using the “+” and “−” buttons of the feedback unit, for example.

### Setting the Focus and Blur Point by the Test Person

The test person may also be asked directly to set the optical power held up to him/her, at which recognition of the directional feature of the adapted visual sign is just barely possible or no longer possible, by himself/herself. This may be done using the “+” and “−” buttons of the feedback unit, for example.

Approaching the Focus and Blur Point from Different Directions

Furthermore, a blur point may be determined when increasing and another blur point when decreasing. These points may be different from each other and subsequently averaged. Alternatively, the sensitivity may be determined as part of a minimization of the error squares using known metrics from both blur points.

### Repeating the Measurement

Of course, the determination of the blurs may also be carried out multiple times to increase the measurement accuracy of the method.

### Monitoring the Accommodation State

During steps 3), 4), 6) or 7), the accommodation state of the at least one eye of the test person may be monitored in the method according to section A or during step 3) or 4) in the method according to section B with the aid of the autorefractometry or aberrometry unit. The results obtained therefrom may be used to control the procedure (e.g. aborting or repeating individual steps in the event of unwanted accommodation (e.g. exceeding a certain threshold)). The measurement may be performed either continuously or only when signaling the recognizability. Furthermore, an accommodation state (sphere, cylinder, lower or higher order aberration) measured—ideally when signaling the recognizability—may be included in the calculation of the sensitivity or refraction deficit.

### E. Blur in the Minus Direction and Inclusion of a Near Measurement

### Blur in the Minus Direction

In the above exemplary embodiments, the applied optical power corresponds to a refraction deficit in the plus direction, since this cannot be compensated for by the test person by accommodation. However, it is also possible to proceed in reverse, i.e. with an applied optical power which corresponds to a refraction deficit in the minus direction. The accommodation which may occur in this case can be dealt with as follows:
Ignoring accommodation;
Measuring test persons who can accommodate, e.g. physiologically (e.g. age-related) or pharmacologically induced (e.g. dripped), or only weakly;
Measuring or monitoring the accommodation state;
Using assumptions about the ability to accommodate (e.g. depending on age according to Duane's curve, see FIG. 3).

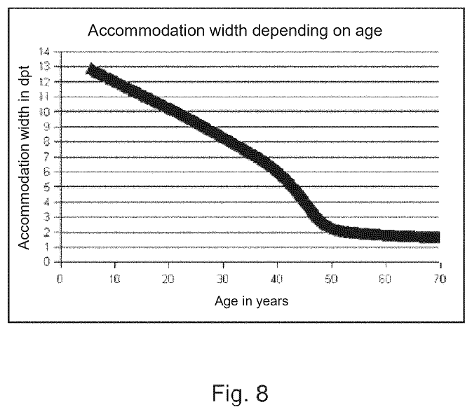

The Duane's curve shown in FIG. 8 is taken from B. Lachenmayr, D. Friedburg, E. Hartmann, A. Buser: “Auge-Brille-Refraktion: Schober-Kurs: verstehen-lernen-anwenden” (“Eye-spectacles-refraction: Schober course: understand-learn-apply”), 2005, FIG. 1.29, and was originally published in Alexander Duane: “Studies in monocular and binocolar accommodation with their clinical applications”, Transactions of the American Ophthalmological Society, vol. 20, 1922, pp. 132-157, PMID 16692582, PMC 1318318. Duane's curve shows that the ability of the human eye to accommodate (accommodation width) decreases continuously from an average of 14 to one diopter from the age of eight to shortly after the age of fifty.

The influence of accommodation on the sphere can be taken into account in the following ways, for example:
The amount of accommodation is subtracted from the amount of the distance of the applied power from the refraction value for distance;
The refraction deficit is calculated directly from the applied power and the measured or assumed refraction value.

In a similar way, the astigmatic deviation may also be calculated using the measured cylinder according to the known formalisms (e.g. cross-cylinder formula, power vector notation) to take into account a change in astigmatism due to accommodation. Furthermore, measured higher order aberrations may be taken into account using known metrics.

### Incorporating a Near Measurement

The procedure described above may be combined with a determination of the objective near refraction values, the maximum accommodation and/or the (lower or higher order) aberrations.

For this purpose, the following procedure may be adopted: The accommodation state of the eye is monitored with (ideally concurrent and as frequent as possible) autorefractometric or aberrometric measurements. It is started with an applied optical power which allows the recognition of the directional feature of the adapted visual sign of the target. This may be an objective, subjective or combined refraction value. In step 5) and possibly in step 6), the optical power held up is then removed from this optical power in the plus direction. In step 4) and possibly in step 7), the test person then signals the time at which he/she can no longer recognize the directional feature of the adapted visual sign. If the applied powers are determined for two visual acuity levels, in this case the applied powers for the higher visual acuity level may be determined first (steps 2-4) and then (steps 5-7) for the lower visual acuity level. In this way, the refraction deficit can be increased in the course of the procedure, whereby first the adapted visual sign with the more difficult recognizability (higher visual acuity level) and then the one with easier recognizability (lower visual acuity level) becomes unrecognizable. The autorefractometric or aberrometric value measured when signaling the loss of recognizability is used to calculate the sensitivity or visual acuity.

The value of the autorefractometric or aberrometric measurement which corresponds to the greatest accommodation is then used as the value (sphere, cylinder, lower or higher order aberration) for the near refraction or for the maximum accommodative power.

### F. Monitoring the Pupil Size

Furthermore, the pupil size (e.g. as a pupil radius) may be monitored, e.g. by means of a camera arranged in the autorefractometer or aberrometer, or by means of a separate camera. The pupil size measured at the blur point or, in a corresponding manner, shortly before (e.g. up to 2 seconds before reaching the blur point) may be used in determining the sensitivity to blur.

The measured pupil size may then be used to quantify the blur of the image on the retina with the aid of a suitably parameterized eye model and the known additional fogging. For example, the angle at which the dispersion disk of a dot shown as blurred can be observed for a given pupil and given additional fogging may be calculated (cf. WO 2019 034525 A1). The sensitivity may be determined in the context of such a visual acuity model as a deterioration in acuity of vision per angle of the dispersion disk.

### G. More Complex Models for Sensitivity

In more complex models, a distinction may be made between the influence of spherical fogging or refraction deficits and astigmatic fogging or refraction deficits. For this purpose, a spherical fogging and an astigmatic fogging may be determined for the same visual acuity level.

I. Combination with Other Measurements

The present invention can be combined very well with other measurements or embedded therein. In a preferred embodiment, the procedure according to sections A or B is performed after an autorefractometric or aberrometric measurement for the distance. Here, this autorefractometric or aberrometric measurement for the distance already represents the first step according to section A and does not have to be performed again. The procedure according to one of the above sections may be performed either before or after any near measurement. The former has the advantage that the (virtual) target is initially still unknown to the test person and the test person has already familiarized themselves with the target for the near measurement.

### LIST OF REFERENCE NUMERALS

V1 first preferential direction
V2 second preferential direction

1-21. (canceled)

22. A method for determining acuity-of-vision characteristics of a test person who has at least one astigmatic visual defect, comprising:
providing visual defect data of the test person, wherein the visual defect data includes at least one cylinder axis or axial position of a required optical cylinder correction;
selecting a preferential direction such that the preferential direction either corresponds to the cylinder axis which is assigned to the optical cylinder correction, or is rotated by 90° to this cylinder axis, or the preferential direction is derived from wavefront data using a point spread function;
applying an optical power at least in the selected preferential direction;
displaying at least one adapted visual sign which has a directional feature, wherein the adapted visual sign is displayed aligned such that its directional feature is arranged in parallel to the preferential direction; and
determining the acuity-of-vision characteristics of the test person for the selected preferential direction, taking into account at least one dimension of the directional feature of the adapted visual sign and the applied optical power.

23. The method according to claim 22, wherein the directional feature of the adapted visual sign comprises a sequence of light and dark areas which follow one another along the preferential direction.

24. The method according to claim 22, comprising:
providing an unadapted standard visual sign having a directional feature;
rotating the standard visual sign in a display plane such that its directional feature is arranged in parallel to the preferential direction; and
displaying the rotated standard visual sign as the adapted visual sign.

25. The method according to claim 22, further comprising:
selecting, as a first preferential direction, the cylinder axis assigned to the optical cylinder correction and arranged in the first principal meridian of the required optical cylinder correction, applying, as the optical power, an optical spherical correction to correct the visual defect of the test person in the first principal meridian according to the visual defect data, and determining, as acuity-of-vision characteristics, the visual acuity of the test person for this first principal meridian, and/or
selecting, as a second preferential direction, a direction rotated by 90° to the cylinder axis arranged in the second principal meridian of the required optical cylinder correction, applying, as the optical power, an optical spherical correction which corrects the visual defect of the test person in the second principal meridian according to the visual defect data, and determining, as acuity-of-vision characteristics, the visual acuity of the test person is determined for this second principal meridian.

26. The method according to claim 25, further comprising:
determining the visual acuity of the test person for both the first and the second principal meridian of the required optical cylinder correction, and deriving a direction-independent visual acuity therefrom.

27. The method according to claim 22, wherein a Landolt ring whose gap is displayed rotated by 90° to the selected preferential direction is used as an adapted visual sign.

28. The method according to claim 22, wherein a Snellen E, in which the connecting line connecting the three parallel E-lines is arranged in parallel to the selected preferential direction, is used as an adapted visual sign.

29. The method according to claim 27, further comprising:
displaying the adapted visual sign at least once rotated clockwise by 90° to the preferential direction and at least once rotated counterclockwise by 90° to the preferential direction; and
asking the test person to distinguish these two differently rotated adapted visual signs from one another as part of a visual task.

30. The method according to claim 22, wherein a hatched area in which the hatching lines are arranged perpendicular to the selected preferential direction is used as an adapted visual sign.

31. The method according to claim 22, further comprising:
displaying, in addition to the adapted visual sign, at least one further visual sign whose gray value corresponds approximately to an averaged gray value of the adapted visual sign; and
asking the test person to distinguish the displayed visual signs from one another as part of a visual task.

32. The method according to claim 22, wherein the applied optical power is varied at least up to a limit refraction for the selected preferential direction from which the test person recognizes the directional feature of the adapted visual sign.

33. The method according to claim 22, wherein the dimension of the directional feature of the adapted visual sign is varied at least up to a limit dimension up to which the test person recognizes the directional feature of the adapted visual sign.

34. The method according to claim 22, further comprising:
ascertaining at least one visual acuity and/or at least one sensitivity and/or at least one visual acuity-refraction value pair and/or at least one refraction value as acuity-of-vision characteristics.

35. The method according to claim 22, wherein the test person is presented with at least one visual task dependent on the displayed adapted visual sign, which the test person answers by giving active and/or passive feedback.

36. The method according to claim 22, further comprising:
determining the visual acuity of the test person in the selected preferential direction at two different applied optical powers, and ascertaining a sensitivity of the test person therefrom.

37. The method according to claim 22, further comprising:
performing a subjective and/or objective refraction, and deriving the visual defect data of the test person from the visual defect of the test person ascertained therein.

38. The method according to claim 22, wherein the visual acuity of the test person is determined as acuity-of-vision characteristics and is converted into a visual acuity type different therefrom.

39. The method according to claim 22, wherein the at least one adapted visual sign is displayed to be presented without correction and/or without complete correction of the optical cylinder correction required by the test person, but sharply for the test person.

40. An apparatus for determining acuity-of-vision characteristics of a test person who has at least one astigmatic visual defect, comprising:
a selection module configured to select a preferential direction, wherein this preferential direction either corresponds to a cylinder axis or axial position which is assigned to an optical cylinder correction required by the test person, or is rotated by 90° to this cylinder axis, or the preferential direction is derived from wavefront data using a point spread function;
a refraction unit configured to apply an optical power to the test person in the selected preferential direction;
a display module having a display and configured to display at least one adapted visual sign having a directional feature on the display such that the directional feature of the adapted visual sign is arranged in parallel to the preferential direction; and
an acuity-of-vision characteristics determination module configured to determine the acuity-of-vision characteristics of the test person for the selected preferential direction, taking into account at least one dimension of the directional feature of the adapted visual sign and the applied optical power.

41. The apparatus according to claim 40, further comprising:
an eye tracking unit configured to track at least one eye of the test person when displaying the at least one adapted visual sign.
